(19) United States
(12) Patent Application Publication
(10) Pub. No.: US 2024/0130935 A1
(43) Pub. Date: Apr. 25, 2024

(54) PROBIOTICS REVITALIZING SYSTEM FOR SKINCARE

(71) Applicant: Nano and Advanced Materials Institute Limited, Hong Kong (HK)

(72) Inventors: Chun Hay KO, Hong Kong (HK); Man Hon YUEN, Hong Kong (HK); Wah Kit CHEUK, Hong Kong (HK); Joo Ann EWE, Hong Kong (HK); Wing Man CHAN, Hong Kong (HK)

(21) Appl. No.: 18/493,751

(22) Filed: Oct. 23, 2023

Related U.S. Application Data
(60) Provisional application No. 63/419,295, filed on Oct. 25, 2022.

(51) Int. Cl. A61K 8/02; A61K 8/36; A61K 8/60; A61K 8/64; A61K 8/73; A61K 8/99; A61Q 19/00

(52) U.S. Cl. CPC A61K 8/0241; A61K 8/361; A61K 8/60; A61K 8/64; A61K 8/736; A61K 8/99; A61Q 19/00; A61K 2800/63; A61K 2800/652; A61K 2800/654

(57) ABSTRACT

An inedible and dry dormant state encapsulated probiotic core-shell particle for external, non-mucosal skin application is provided. From inside out, the core-shell particle has a structure of a carrier particle core serving as a nutrient source for probiotics, a first layer including a dormant probiotic species for affecting epidermal biome and at least one prebiotic as a food source for the probiotic, a polymer layer positioned over the first layer, and a dissolvable protective layer for protecting the probiotic core-shell particle from oxidation, heat and humidity. By co-applying with a releasing medium, the dissolvable protective layer and the polymer layer are dissolved to expose the dormant probiotic containing layer, the first layer. Further, the releasing medium also is able to activate and reconstitute the dormant probiotic to a live probiotic on the applied non-mucosal epidermal surface.

### CROSS-REFERENCE TO RELATED APPLICATIONS

The present application claims priority from U.S. provisional patent application Ser. No. 63/419,295 filed Oct. 25, 2022, and the disclosure of which is incorporated herein by reference in its entirety.

### FIELD OF THE INVENTION

The present invention generally relates to probiotic fields. More specifically the present invention relates to a dormant state encapsulated probiotic core-shell particle and a technology to reactivate the dormant probiotic for external application.

### BACKGROUND OF THE INVENTION

Probiotics are living organisms which upon ingestion in certain numbers exert health beneficial effects beyond inherent general nutrition. Scientific and commercial interest on probiotics as well as their effects on human health has been increasing since last decade. In recent years, the rapid increase in medical use of probiotics has confirmed their safety as human health modulator. There are increasing number of studies showing probiotics exert additional health-promoting effects on other parts of human body besides digestive system, such as skin health. Being the “good bacteria” that naturally colonizes in human body, the potential use of probiotics in maintaining skin health under healthy and inflammatory conditions, such as atopic dermatitis and acne skin, has been in the limelight in pharmaceutical, skincare and healthcare industries. Gut microbiome modulation through fecal transplant has been proven to be a valid therapeutic strategy in diseases such as Clostridium difficile infections. Therefore, modulation of skin microbiome may be an interesting therapeutic approach to improve skin condition. Nevertheless, there are only limited studies on healthy subjects to show a beneficial effect of probiotics on skin health, while most of them demonstrated probiotics are effective in dealing with problematic skins. Non-viable bacterial products or metabolic lysates from probiotics instead of live probiotics without proper formulation, are used in most of the studies. Furthermore, combinations/formulations of probiotics, and their mechanism of action, such as alteration in microbiota composition or function (dysbiosis) on healthy and problematic skins remain to be fully elucidated.

Moreover, the degree of probiotics colonization on skin surface affects the actual and long-term beneficial effects provided by the live probiotics. Probiotics are highly sensitive to their growing environment, particularly the skin microenvironment that is highly variable due to endogenous host factors and exogenous environmental factors. Any newly introduced live probiotics will have difficulty surviving on the skin surface if no suitable measures are involved, such as creating an adaptation site or nutrient/moisture-rich environment; further, they will not be able to colonize on the skin. Thus, to increase the colonization rate of probiotics is another challenge.

Recently, skincare products with bacteria lysate predominate the probiotics-related cosmetic industry. The major reason is that common skincare products are not conducive to the survival of probiotics due to the preservatives and water-rich environment. Live probiotics require refrigeration to extend their shelf-life which hinders their distribution and storage. Thus, there is still a lack of live probiotics skincare/cosmetic products in the market. The present invention addresses this need.

### SUMMARY OF THE INVENTION

It is an objective of the present invention to provide a live probiotics form skincare/cosmetic additive that maintains a long-term beneficial effect to dysbiosis skin, and it is able to survive through unfavorable conditions and maintain the survival in skincare products without extra needs of exclusive storage condition.

In accordance with a first aspect of the present invention, the present invention provides an inedible and dry dormant state encapsulated probiotic core-shell particle for external, non-mucosal skin application. The particle includes a carrier particle core serving as a nutrient source of probiotics, a first layer surrounding the carrier particle core and having at least one dormant probiotic species for affecting a non-mucosal, external epidermal biome and at least one prebiotic as a food source for the probiotic, a polymer layer positioned over the first layer, and a dissolvable protective layer for protecting the probiotic core-shell particle from oxidation, heat and humidity. Noteworthy, the dormant probiotic in the particle can be activated and reconstituted to a live probiotic when mixed with a corresponding releasing medium to dissolve the dissolvable protective layer on a non-mucosal epidermal surface.

In accordance with one embodiment of the present invention, the carrier particle core includes one or more of sucrose, whey protein, starch and cellulose.

In accordance with another embodiment of the present invention, the at least one probiotic species includes Bifidobacterium, Lactobacillus, Lactococcus, Leuconostoc, Streptococcus, Enterococcus, Staphylococcus, Saccharomyces, Kluyveromyces, and the strain variants thereof.

In accordance with one embodiment of the present invention, the at least one prebiotic is selected from a protein or a saccharide. In some embodiments, the saccharide includes a polysaccharide, a monosaccharide or a disaccharide.

In accordance with one embodiment of the present invention, the protein is selected from a whey protein, a casein protein, a soy protein, a milk protein, a pea protein, a rice protein, a zein, or a bovine serum albumin.

In accordance with another embodiment of the present invention, the saccharide is selected from dextrose, fructose, galactose, sucrose, lactose, maltose, trehalose, cellobiose, chitobiose, dextrin, maltodextrin, cyclodextrin, xylitol, cellulose, chitin, chitosan, pullulan, pectin, alginates or arabinoxylans.

In accordance with one embodiment of the present invention, the polymer layer is selected from shellac, dipalmitoyl hydroxylproline, a methacrylate-based polymer or copolymer, a glyceride, or poly-L-lactic acid.

In accordance with one embodiment of the present invention, the dissolvable protective layer includes a polymer, a surfactant, a fatty acid and a mineral. In some embodiments, the surfactant is selected from an anionic surfactant or a non-ionic surfactant. In some embodiments, the fatty acid is selected from palmitic acid, stearic acid, oleic acid, linoleic acid, arachidonic acid, eicosapentaenoic acid or docosahexaenoic acid. In some embodiments, the mineral is selected from talc, kaolin, ZnO, TiO₂ or SiO₂.

In accordance with one embodiment of the present invention, the inedible and dry dormant state encapsulated probiotic core-shell particle includes 67.2% to 92.19% of carrier particle core, 0.01% to 0.1% of probiotics, 0% to 14.7% of polymers, 7.8% to 11.5% of saccharides, 0% to 2.2% of proteins, 0% to 1% of fatty acids, 0% to 1.3% of surfactants and 0% to 2.1% of minerals.

In accordance with a second aspect of the present invention, the present invention provides a topically applied kit for modulating a microbiome of a non-mucosal epidermis area. The kit includes a plurality of the dormant, encapsulated probiotic core-shell particles as described above and a releasing medium, for reconstituting the dormant, encapsulated probiotic core-shell particles. The releasing medium is able to degrade the protective layer and the polymer layer of the dormant encapsulated probiotic core-shell particles to convert the dormant probiotic to an activated, live probiotic and further form a synthetic biofilm including the activated, live probiotic on the non-mucosal epidermis providing a microenvironment for probiotics colonization.

In accordance with one embodiment of the present invention, the releasing medium includes water, a salt, an organic acid, a surfactant, an oil and a film forming nutrient. In some embodiments, the salt is selected from NaCl or CaCl₂. In some embodiments, the organic acid is selected from an acetic acid, a lactic acid or a citric acid. In some embodiments, the surfactant is selected from is selected from an anionic surfactant or a non-ionic surfactant. In some embodiments, the oil is selected from a squalane, a meadowfoam seed oil, a soybean oil, an isopropyl myristate, an isopropyl palmitate, a paraffin oil, an almond oil or a soybean oil. In some embodiments, film forming nutrient is selected from a hyaluronic acid or an extracellular polysaccharide.

In accordance with one embodiment of the present invention, the releasing medium includes 95% to 99.98% of water, 0% to 0.9% of salts, 0% to 0.02% of organic acids, 0.01% to 2% of surfactants, 0.01% to 1% of oils and 0% to 0.9% of film forming nutrients.

In accordance with another embodiment of the present invention, the releasing medium is selected from a lotion form, a cream form, a serum form, or a solution form.

In accordance with one embodiment of the present invention, the releasing medium further includes a postbiotics agent possessing antibacterial effect and anti-inflammatory property.

In accordance with another embodiment of the present invention, the postbiotics agent includes a lysate or a ferment of the probiotic same as the vehicle and contains probiotic cell wall debris, growth metabolites, and dead probiotic cell.

In accordance with one embodiment of the present invention, the postbiotics agent includes 0.01% to 0.1% of postbiotics materials and 99.8% to 99.99% of saccharides.

In accordance with a third aspect of the present invention, the present invention provides a method of maintaining skin health by modulating skin microbiome. The method includes topically applying the above-mentioned kit to a non-mucosal external epidermal area in need thereof.

In accordance with one embodiment of the present invention, the skin area in need thereof suffers from inflammation, dehydration, acne, infection, and reddening.

In accordance with a fourth aspect of the present invention, the present invention provides a method of manufacturing inedible and dry dormant state encapsulated probiotic core-shell particles for external, non-mucosal skin application. Particularly, the method includes the following steps:
preparing a carrier particle core comprising one or more nutrient sources for probiotics; coating the carrier particle core with a first layer comprising dormant probiotics and at least one prebiotic, forming a core-shell structure;
applying a polymer layer over the first layer;
depositing a dissolvable protective layer over the polymer layer; and drying and conditioning the resulting particles to create inedible and dry dormant state encapsulated probiotic core-shell particles.

### BRIEF DESCRIPTION OF THE DRAWINGS

Embodiments of the invention are described in more details hereinafter with reference to the drawings, in which:

FIG. 1 depicts the probiotics' antibacterial effects determined by evaluating inhibition zone;

FIGS. 2A-2C demonstrate the anti-inflammatory effect of Lactobacillus probiotics on human skin keratinocyte cell line; FIG. 2A shows the anti-inflammatory effect of Lactobacillus plantarum (JCM 6651); FIG. 2B shows the anti-inflammatory effect of Lactobacillus johnsonii (JCM 1101); and FIG. 2C shows the anti-inflammatory effect of Lactobacillus reuteri (JCM 1084);

FIG. 3 shows the anti-inflammatory effect of Lactobacillus probiotics on human 3D skin model;

FIGS. 4A-4D show the proliferative effect of different prebiotics supplementation in different concentrations on Lactobacillus probiotics; FIG. 4A demonstrates that whey protein supplementation increases the growth of Lactobacillus plantarum (JCM 6651) by 21862%;

FIG. 4B depicts that xylitol supplementation increases the growth of Lactobacillus johnsonii (JCM 1101) by 496%; FIG. 4C shows that whey protein has the best performance at 1%; and FIG. 4D shows that xylitol has the best performance at 1%;

FIG. 5 depicts the manufacture process of the dormant state encapsulated probiotic core-shell particle;

FIGS. 6A-6D demonstrates the SEM morphology of the particle at each manufacture step; FIG. 6A depicts the SEM image of the carrier particle core; FIG. 6B is the SEM image of the carrier particle core coated with the first layer; FIG. 6C shows the SEM image of the polymer layer on the first layer surface; and FIG. 6D depicts the SEM image of the dormant state encapsulated probiotic core-shell particle after the dissolvable protective layer is coated on the polymer layer;

FIG. 7 depicts the probiotics viability and water activity of DP17 for 24 weeks;

FIG. 8 depicts the probiotics viability and water activity of DP18 for 24 weeks;

FIG. 9 shows the probiotics viability and water activity of DP19 for 24 weeks;

FIG. 10 demonstrates the probiotics viability and water activity of DP20 for 24 weeks;

FIG. 11 shows the antibacterial performance of postbiotics;

FIG. 12 demonstrates probiotics colonization and skin hydration evaluation process;

FIG. 13 shows the probiotics amount on skin before and after applying PRS; and

FIG. 14 shows the skin hydration level before and after applying PRS.

### DETAILED DESCRIPTION

In the following description, an inedible and dry dormant state encapsulated probiotic core-shell particle for external, non-mucosal skin application and the like are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Creating the dormant state encapsulated probiotic core-shell particles involves a meticulous selection process of probiotic species. Unlike mucosal surfaces, the skin's unique environmental conditions limit the range of microbial types capable of thriving in its harsh milieu, primarily favoring Gram-positive species. The skin harbors both resident and transient microbial populations. “Resident” species denote viable, self-sustaining communities, while “transient species” are typically contaminants with limited or no capacity for prolonged growth and reproduction in the cutaneous milieu. Among the resident microbial species are Propionibacterium (including P. acnes, P. avidum, and P. granulosum), coagulase-negative Staphylococcus (such as Staphylococcus epidermidis), Micrococcus, Corynebacterium, Acinetobacter, Malassezia yeast species, and various bacteriophage species. In contrast, common transient species encompass Staphylococcus aureus, Escherichia coli, Pseudomonas aeruginosa, and Bacillus species. Resident species typically serve as commensals, generally posing no harm to the host while actively competing against transient and pathogenic bacteria through antimicrobial factors or impeding colonization. Disruptions in the skin's microbiome can pave the way for transient and opportunistic species to colonize, potentially leading to disease. Consequently, the introduction of topically applied effective probiotics may directly influence the skin's microbiome. Given that probiotic functions are highly strain-specific, probiotic species are exclusively evaluated with a track record of safety and documented skin-health promotion, excluding any species associated with severe health issues, for their antibacterial and anti-inflammatory effects.

To address the challenges outlined above, the present invention introduces a novel technology for dormant probiotics, resulting in the development of a probiotic revitalizing system (PRS). This system consists of inedible and dry dormant state encapsulated probiotic core-shell particles and a releasing medium. It serves to shield probiotics from unfavorable conditions and safeguard their viability within commercial products. The dormant probiotics technology functions by maintaining probiotics in a dormant state until application. When these dormant probiotics come into contact with a compatible releasing medium on the skin and combine with a synthetic biofilm, they become activated, ensuring proper colonization. Those probiotics renowned for their skin-health-promoting effects are meticulously evaluated, particularly assessing their antibacterial and anti-inflammatory properties. The most effective probiotics are then encapsulated using biopolymers and/or prebiotics to preserve their viability, resulting in the formulation of dormant state encapsulated probiotic core-shell particles. A corresponding formulation for the releasing medium is devised to facilitate the discharge and enrichment of probiotics while forming a protective layer. These probiotics formulations are subjected to rigorous evaluation by accredited third-party entities to assess product safety and performance. They can be integrated into various cosmetic products, including creams, serums, and lotions, to function effectively even in adverse environmental conditions.

As used herein, the term of “dormant probiotic” refers to a live beneficial microorganism, typically of the Lactobacillus species, that has been rendered inactive or dormant through encapsulation within a protective structure, such as a core-shell particle. In this state, the probiotic remains viable but inactive until conditions are suitable for reactivation.

As used herein, the term of “prebiotic” refers to a substance, often a carbohydrate like maltodextrin, xylitol, or saccharides, that serves as food or nourishment for probiotics. Prebiotics are included in the formulation to promote the growth and activity of probiotics.

As used herein, the term of “dissolvable protective layer” refers to a coating surrounding the dormant probiotics within the core-shell particle. This layer is designed to dissolve when exposed to a releasing medium, thereby allowing the probiotics to become active and available for use.

As used herein, the term of “releasing medium” refers to a formulated solution designed to dissolve the protective layers of the core-shell particles and reactivate the dormant probiotics. It often contains organic acids, film-forming nutrients, surfactants, oils, and other components to facilitate probiotic release and colonization upon application to the skin.

As used herein, the term of “synthetic biofilm” refers to a created structure made from combinations of polysaccharides, proteins, and water-retaining ingredients, such as cellulose, chitosan, pullulan, whey protein, casein, amino acids, hyaluronic acid, and saccharide isomerate. It is used as a substrate for probiotics to adhere to and colonize when applied to the skin. This biofilm simulates the natural conditions for probiotics.

As used herein, the term of “postbiotics agent” refers to a postbiotics agent refers to a formulation composed of cell lysates, growth metabolites, and cellular debris from probiotics. These substances are typically derived from probiotics that have been lysed or broken down. Postbiotics can also include prebiotics. The formulation is used for its beneficial effects on the skin, such as anti-inflammatory and antibacterial properties, without requiring live probiotics.

In accordance with a first aspect of the present invention, an inedible and dry dormant state encapsulated probiotic core-shell particle for external, non-mucosal skin application is provided. The survival of probiotics in a product is affected by several factors such as pH, post-acidification during products fermentation, hydrogen peroxide production, oxygen and storage temperature. Minor changes in these factors will cause the probiotics to lose its viability, despite being sustained in nutrient rich or its niche environment. A dormant probiotic technology demonstrated by the present invention is developed to manufacture a dormant state encapsulated probiotic core-shell particle for delivering live and active probiotics with enhanced adhesion properties in skincare products that are otherwise generally unfavorable to support the growth of microorganisms.

The dormant state encapsulated probiotic core-shell particle includes a carrier particle core serving as a nutrient source of probiotics, a first layer surrounding the carrier particle core and having at least one dormant probiotic species for affecting a non-mucosal, external epidermal biome and at least one prebiotic as a food source for the probiotic, a polymer layer positioned over the first layer, and a dissolvable protective layer for protecting the probiotic core-shell particle from oxidation, heat and humidity. By encapsulating dormant probiotics in the core-shell particle, the probiotics survive within those protective coatings, including the polymer layer and the dissolvable protective layer, which protect the probiotics from moisture, oxygen and harmful substances. The formulation of the particle is performed by screening the different types of biopolymers, polysaccharides, lipids or proteins, such as poly-L-lactic acid, polymethyl methacrylate, pectin, sodium alginate, chitosan, zein, bovine serum albumin, stearic acid and paraffin oil, to provide protective layers for the probiotics.

In the present formulations, examples of the carrier particle core include one or more of sucrose, whey protein, starch and cellulose sphere, and the probiotic species may be Bifidobacterium, Lactobacillus, Lactococcus, Leuconostoc, Streptococcus, Enterococcus, Staphylococcus, Saccharomyces, Kluyveromyces, or the strain variants thereof; further, examples of the polymer layer include shellac, dipalmitoyl hydroxylproline, a methacrylate-based polymer or copolymer, a glyceride, or poly-L-lactic acid. The dissolvable protective layer include a polymer, a surfactant, a fatty acid and a mineral. Exemplary surfactant includes an anionic surfactant and a non-ionic surfactant, and exampled fatty acid includes palmitic acid, stearic acid, oleic acid, linoleic acid, arachidonic acid, eicosapentaenoic acid and docosahexaenoic acid; further, examples of mineral include talc, kaolin, ZnO, TiO₂ and SiO₂.

Prebiotics are also a part of the encapsulation materials for the formation of the protective coating, which is able to withstand moisture, pH and contain the probiotics within the microsphere and thus enable the probiotics to survive upon long term storage. Prebiotics, once defined as “non-digestible food ingredients that beneficially affect the host by selectively stimulating the growth and/or activity of one, or a limiting number of, bacteria in the colon”, has been re-defined recently as “a substrate that is selectively utilized by host microorganisms conferring a health benefit” due to its extended application beyond gut health. Prebiotics not only have protective effects on the gastrointestinal system but also on other parts of the body, including the skin. The prebiotics include a protein and a saccharide. Examples of the protein include a whey protein, a casein protein, a soy protein, a milk protein, a pea protein, a rice protein, a zein, or a bovine serum albumin, and examples of the saccharide include a polysaccharide, a monosaccharide and a disaccharide, such as dextrose, fructose, galactose, sucrose, lactose, maltose, trehalose, cellobiose, chitobiose, dextrin, maltodextrin, cyclodextrin, xylitol, cellulose, chitin, chitosan, pullulan, pectin, alginates and arabinoxylans.

To prepare the particles, a freeze-drying procedure is chosen to process and transform the live form probiotics to a dormant state. Freeze-drying, also known as lyophilization or cryodesiccation, is a low temperature dehydration process that involves freezing the product, lowering pressure, then removing the ice by sublimation. Subjecting probiotics to lyophilization induces a state of dormancy to the probiotics where the cellular metabolism is completely halted without a change in the physiological and genetic features. Lyophilization is always preceded by a cryopreservation process, where the probiotics are subjected to a cryogenic temperature (−80° C.) which promotes ice crystal formation in the suspension medium and within the cell interior causing cryo-injuries. Cryoprotectants such as glycerol are used to protect the probiotics from cryo-injuries during cryopreservation. During cryopreservation, the biochemical and physiological activities of the probiotics are essentially halted, and cells can be protected for long periods of time until resuscitation. This method is used to produce probiotics or postbiotics formulations in powder form, which contain temperature sensitive probiotics or temperature sensitive proteins. In brief, the solution of probiotics or postbiotics formula is frozen in a −80° C. refrigerator or with liquid nitrogen. Then the frozen sample is transferred into the allocated vacuum environment in a freeze dryer to remove the ice water through sublimation. The freeze-dried dormant state probiotics are in powder form after the whole freeze-drying process.

After the preparation of dormant state probiotics powder, fluidized bed coating is further conducted to make core-shell particles. Fluidized-bed coating is a method to form a coating on granules or particles. This is used to form the protective layers, including the polymer layer and the dissolvable protective layer, of the dormant state probiotics core-shell particle. In fluidized-bed coating, the carrier particle core is first spray-coated with a mixture made by mixing the dormant state probiotics powder and the prebiotics solution to form the first layer. After that, the carrier particle core with the first layer is fluidized by vertical air flow through a distributor plate at the bottom of the system. The coating material, which can be a melt, a suspension or a solution, is sprayed onto the fluidized particle, where the single droplets impact the particle surface and spread, whereas the solvent evaporates constantly. The remaining solid component deposits on the particle surface and forms a shell of layers, which causes particle growth. The aim of this process is the homogeneous deposition of droplets on a single particle and over an entire particle population, which is critical to achieve a homogeneous coating layer thickness. After the mixture is dried to from a first protective layer, the polymer layer, the second protective layer is also prepared by spray-coated with another solution to form the dissolvable protective layer. With a proper drying, the inedible and dry dormant state encapsulated probiotic core-shell particle is obtained.

In accordance with a second aspect of the present invention, a topically applied kit for modulating a microbiome of a non-mucosal epidermis area is further provided. The kit includes a plurality of the dormant, encapsulated probiotic core-shell particles and a releasing medium, for reconstituting the dormant, encapsulated probiotic core-shell particles. The releasing medium is able to degrade the protective layer and the polymer layer of the dormant encapsulated probiotic core-shell particles to convert the dormant probiotic to an activated, live probiotic and to further form a synthetic biofilm including the activated, live probiotic on the non-mucosal epidermis providing a microenvironment for probiotics colonization.

The synergy between the dormant, encapsulated probiotic core-shell particles and the releasing medium plays a pivotal role in releasing the probiotics that are shielded by the polymer layer and the dissolvable protective layer. The composition of the releasing medium is tailored to the materials of the protective layer, enabling it to dissolve the particle shells and liberate the probiotics held within. Moreover, the rate of probiotic release from the particles can be meticulously controlled through the formulation of the releasing medium. The combined application of these particles and the releasing medium exclusively occurs at the skin surface, ensuring the delivery of live probiotics.

Beyond triggering the release of live probiotics from the dormant probiotic core-shell particles, the releasing medium also establishes a conducive microenvironment for probiotic colonization on the skin. Conventional probiotics, such as Lactobacillus, are not natural commensal microbes on the skin, presenting a challenge for non-commensal probiotics to establish themselves on the skin's surface. To address this, a film-forming nutrient is introduced into the releasing medium, creating a synthetic biofilm. This biofilm aids probiotics in adapting to their new environment by providing essential nutrients, moisture, and adhesion sites in the form of extracellular polymeric saccharides. The synthetic biofilm environment expedites probiotic colonization and adaptation. Importantly, the formulation incorporates natural biofilm materials that do not harm the microorganisms present on the applied surface but instead foster a fresh and suitable microenvironment for probiotics to reactivate, flourish, and reproduce. The probiotics releasing medium can also be further developed and adapted into various product forms, including lotions, creams, and serums.

The releasing medium is formulated by screening different surfactants, acids, alkalis and solvents, such as Tween 20, Tween 60, Span 20, Span 60, Brij 30, Brij 35, citric acid, acetate acid, lactic acid, sodium hydroxide, potassium hydroxide, triethanolamine, alcohol, butanediol and PEG, to form a nano to micro sized emulsion for dissolving the protective coating of the core-shell particle to release the inside probiotics. The formulations of releasing medium include water, a salt, an organic acid, a surfactant, an oil and a film forming nutrient. The exemplary surfactants include anionic surfactants and non-ionic surfactants, the examples of salt include NaCl and CaCl₂, the exampled organic acids include acetic acid, lactic acid and citric acid, the examples of oil include squalane, meadowfoam seed oil, soybean oil, isopropyl myristate, isopropyl palmitate, paraffin oil, almond oil and soybean oil, and the exampled film forming nutrient includes hyaluronic acid and extracellular polysaccharide.

The effectiveness of the releasing medium's probiotics-releasing capability is assessed through a probiotics-releasing profile test. Various formulations of film-forming nutrients are screened, incorporating diverse combinations of polysaccharides, proteins, and moisture-retaining ingredients such as cellulose, chitosan, pullulan, whey protein, casein, amino acids, hyaluronic acid, and saccharide isomerate. These formulations are designed to create a synthetic biofilm conducive to the adoption and colonization of probiotics. Evaluation is conducted using either artificial skin or porcine skin, quantifying the number of probiotics that successfully colonize and persist on the tested surface over a specified period.

Additionally, the releasing medium includes a safeguarded postbiotics agent. Given that skincare products featuring bacteria lysates and/or ferments, collectively known as postbiotics due to their content of pathogen-inhibitory substances like bacteriocins and organic acids, have become prevalent in the probiotics-related cosmetic industry, the present invention leverages the cell lysates/ferment of the probiotic strain exhibiting the most potent antibacterial and anti-inflammatory properties to create a safeguarded postbiotics formulation. Probiotic cell lysates are prepared by subjecting fermented cultures to thermal treatment or sonication, causing the cell membranes to rupture. These cell lysates and ferments contain remnants of probiotic cell walls, growth metabolites, and deceased probiotics from the same probiotic strain as the carrier.

In accordance with a third aspect of the present invention, a method of maintaining skin health by modulating a skin microbiome is provided. The method includes topically applying the above-mentioned kit to a non-mucosal external epidermal area suffering from inflammation, dehydration, acne, infection, and reddening.

The occurrence of skin diseases such as atopic dermatitis and acne vulgaris has been suggested to be associated with disruption of normal microbiota. Therefore, the modulation of skin microbiome by re-establishing a beneficial bacteria microbiome is able to alleviate these diseases and subsequently promote skin health. Probiotics have been reported to possess bacterial inhibition ability and promote positive effects to the skin microenvironment balance through several underlying mechanisms such as production of inhibitory substances such as bacteriocins and organic acids as well as binding sites competition. Therefore, the application of probiotics and/or its metabolites to inhibit pathogens colonization facilitates the re-establishment of a balanced skin microbiome.

In accordance with a fourth aspect of the present invention, a method of manufacturing inedible and dry dormant state encapsulated probiotic core-shell particles for external, non-mucosal skin application is provided.

The method begins with the preparation of a carrier particle core that serves as a rich source of nutrients to sustain probiotic vitality. This core acts as the foundational structure upon which the probiotic particles are built. The next step involves enveloping this carrier particle core with a first layer meticulously engineered to house dormant probiotics. This first layer is not just a protective shield but also a nourishing environment for the probiotics. In addition to the dormant probiotics, it incorporates at least one prebiotic, creating a synergistic core-shell structure that promotes probiotic health and vitality. Following the formation of the core-shell structure, a polymer layer is applied over the first layer. This polymer layer serves as an additional protective barrier, safeguarding the probiotics from external environmental factors, including humidity and oxidation. To ensure optimal probiotic preservation and release, a dissolvable protective layer is deposited atop the polymer layer. This layer acts as a safeguard until the probiotics are ready for activation. It dissolves when it encounters the appropriate releasing medium, facilitating the probiotics' release and subsequent activation. The final step in this manufacturing process involves drying and conditioning the resultant particles. This crucial phase creates inedible and dry dormant state encapsulated probiotic core-shell particles that are primed for external, non-mucosal skin application. These particles are carefully crafted to retain the probiotics' vitality and efficacy, ensuring that they remain effective when applied to the skin's external surface.

### Examples

### Example 1. Probiotic Screening: Finding the Probiotic Species that Possesses the Best Beneficial Effects

Preparation of Probiotics

The frozen probiotics seed stock, comprising a single, pure strain, is transferred into the bioreactor fermentation vessel along with a culture medium designed to facilitate growth. Temperature control is maintained by connecting a refrigerated circulator to the bioreactor fermentation vessel. The culture temperature is carefully maintained within the range of 30° C. to 37° C., while the duration of the culture process spans from 24 to 72 hours, contingent upon the type and strain of the probiotics under examination. The provision of either an aerobic or anaerobic environment is determined based on the specific characteristics of the probiotics.

Subsequent to the fermentation process, the probiotics are harvested through centrifugation, resulting in the formation of a probiotics pellet. This pellet is meticulously collected and then subjected to an antibacterial test. To minimize the risk of contamination, all samples are diligently handled within a biosafety cabinet or a laminar flow chamber.

TABLE 1
Tested probiotics

| | Strain | |
|---|---|---|
| Species | Number | Probiotics |
| Pediococcus | JCM2014 | Pediococcus acidilactici |
| | JCM2032 | Pediococcus acidilactici |
| | JCM8789 | Pediococcus acidilactici |
| | JCM20119 | Pediococcus acidilactici |
| | JCM20076 | Pediococcus pentosaceus |
| Lactococcus | JCM16167 | Lactococcus lactis subsp. cremoris |
| | JCM20101 | Lactococcus lactis subsp. lactis |
| | JCM20128 | Lactococcus lactis subsp. lactis |
| | NZ9100 | Lactococcus lactis |
| Leuconostoc | JCM1564 | Leuconostoc mensenteroides subsp. mensenteroides |
| | JCM6124 | Leuconostoc mensenteroides subsp. mensenteroides |
| | JCM11042 | Leuconostoc mensenteroides subsp. mensenteroides |
| | JCM11043 | Leuconostoc mensenteroides subsp. mensenteroides |
| | JCM9700 | Leuconostoc mensenteroides subsp. dextranicum |
| | JCM20317 | Leuconostoc mensenteroides subsp. dextranicum |
| Lactobacillus | JCM 1084 | Lactobacillus reuteri |
| | JCM 1112 | Lactobacillus reuteri |
| | JCM 1081 | Lactobacillus reuteri |
| | JCM 1091 | Lactobacillus curvatus |
| | JCM 1096 | Lactobacillus curvatus |
| | JCM 1002 | Lactobacillus delbrueckii subsp. Bulgaricus |
| | JCM 1001 | Lactobacillus delbrueckii subsp. Bulgaricus |
| | JCM 20398 | Lactobacillus delbrueckii subsp. Bulgaricus |
| | JCM 11125 | Lactobacillus plantarum/arizonensis |
| | JCM 1149 | Lactobacillus plantarum subsp. plantarum |
| | JCM 6651 | Lactobacillus plantarum subsp. plantarum |
| | JCM 1101 | Lactobacillus johnsonii |
| | JCM 1096 | Lactobacillus curvatus |
| | JCM 1091 | Lactobacillus curvatus |
| | JCM 1133 | Lactobacillus casei subsp alactosus |
| | JCM 1181 | Lactobacillus casei subsp. pseudoplantarum |
| | JCM 1161 | Lactobacillus casei subsp. pseudoplantarum |
| | JCM8129 | Lactobacillus casei |
| | JCM 1170 | Lactobacillus brevis |
| | JCM 1059 | Lactobacillus brevis |
| | JCM 1061 | Lactobacillus brevis |
| | JCM 1084 | Lactobacillus reuteri |
| | KCTC 1120 | Lactobacillus johnsonii |
| | KCTC 3102 | Lactobacillus brevis |
| | KCTC 3141 | Lactobacillus johnsonii |
| | KCTC 3144 | Lactobacillus gasseri |
| | KCTC3148 | Lactobacillus gasseri |
| | KCTC 3163 | Lactobacillus gasseri |
| | KCTC 3181 | Lactobacillus gasseri |
| | KCTC 3237 | Lactobacillus rhamnosus (Lactobacillus casei subsp |
| | | rhamnosus) |
| | KCTC 3510 | Lactobacillus paracasei subsp paracasei |
| | KCTC 5049 | Lactobacillus fermentum |
| Streptococcus | JCM20026 | Streptococcus thermophiles |
| | JCM17834 | Streptococcus thermophilus |
| Enterococcus | JCM5803 | Enterococcus faecalis |
| | JCM7783 | Enterococcus faecalis |
| | JCM8727 | Enterococcus faecalis |
| Staphylococcus | JCM2414 | Staphylococcus epidermidis |
| | JCM5692 | Staphylococcus epidermidis |
| | JCM5693 | Staphylococcus epidermidis |
| | JCM20345 | Staphylococcus epidermidis |
| Saccharomyces | JCM1499 | Saccharomyces cerevisiae |
| | JCM1817 | Saccharomyces cerevisiae |
| | JCM1819 | Saccharomyces cerevisiae |
| | JCM7255 | Saccharomyces cerevisiae |
| Kluyveromyces | JCM5219 | Kluyveromyces lactis |
| | JCM9563 | Kluyveromyces lactis |
| | JCM22014 | Kluyveromyces lactis |

Antibacterial Performance

The antibacterial effectiveness of the probiotics is assessed through their interaction with skin-infective bacteria, such as Staphylococcus aureus. In a nutshell, the agar overlay technique is employed to investigate the probiotics' capacity to inhibit bacterial growth. Initially, the surface of MRS agar is spot-inoculated with 2 μL of an overnight culture of the probiotics under examination, such as lactobacilli. The optical density (OD) of this culture is adjusted to 1.0±0.02 at 550 nm. Each dish receives three spot inoculations.

The agar plates are then incubated at 37° C. for a 24-hour period to facilitate colony development in spot form. Subsequently, they are overlaid with soft Muller-Hinton agar, which consists of 0.8% agar and is pre-mixed with the skin pathogen to be tested, in this case, Staphylococcus aureus. The optical density of the Staphylococcus aureus culture is also adjusted to 1.0±0.02 at 550 nm. Following this step, the plates are incubated at 37° C. for an additional 48 hours in a binder incubator, during which the overlaid agar medium solidifies.

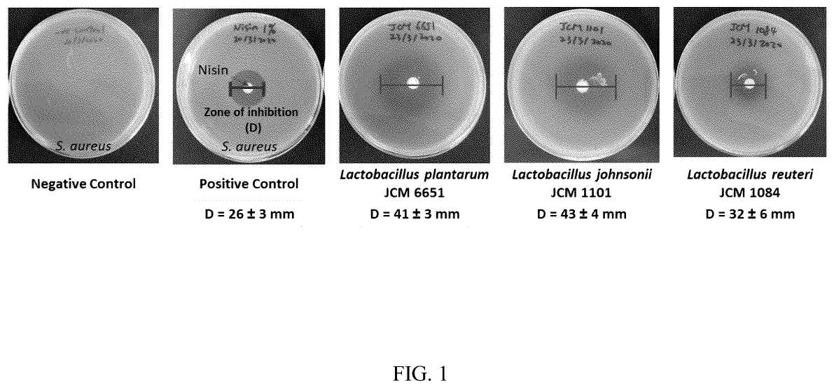

As depicted in FIG. 1, the formation of a clear zone around the probiotics colony, for instance, lactobacilli, is documented as a positive sign of inhibition, and the diameter (measured in millimeters) of this inhibition zone is carefully recorded. The results, indicating the relative zone of inhibition compared to a positive control, are presented in Table 2.

TABLE 2
Probiotics tested with antibacterial effect (against Staphylococcus aureus).

| | | | Antibacterial Test |
|---|---|---|---|
| | | | against SA |
| | | | (Relative zone of |
| | | | inhibition compared |
| | Strain | | to positive control, |
| Species | Number | Probiotics | 1% nisin = 1) |
| Pediococcus | JCM2014 | Pediococcus acidilactici | 1.94 ± 0.00 |
| | JCM2032 | Pediococcus acidilactici | 1.80 ± 0.03 |
| | JCM8789 | Pediococcus acidilactici | 1.88 ± 0.06 |
| | JCM20119 | Pediococcus acidilactici | 1.78 ± 0.12 |
| | JCM20076 | Pediococcus pentosaceus | 1.86 ± 0.12 |
| Lactococcus | JCM16167 | Lactococcus lactis subsp. | 1.48 ± 0.11 |
| | | cremoris | |
| | JCM20101 | Lactococcus lactis subsp. lactis | 1.44 ± 0.11 |
| | JCM20128 | Lactococcus lactis subsp. lactis | 1.58 ± 0.03 |
| | NZ9100 | Lactococcus lactis | 0.95 ± 0.03 |
| Leuconostoc | JCM1564 | Leuconostoc mensenteroides | 1.78 ± 0.10 |
| | | subsp. mensenteroides | |
| | JCM6124 | Leuconostoc mensenteroides | 1.87 ± 0.10 |
| | | subsp. mensenteroides | |
| | JCM11042 | Leuconostoc mensenteroides | 1.98 ± 0.15 |
| | | subsp. mensenteroides | |
| | JCM11043 | Leuconostoc mensenteroides | 1.59 ± 0.02 |
| | | subsp. mensenteroides | |
| | JCM9700 | Leuconostoc mensenteroides | 1.57 ± 0.06 |
| | | subsp. dextranicum | |
| | JCM20317 | Leuconostoc mensenteroides | 1.51 ± 0.06 |
| | | subsp. dextranicum | |
| Lactobacillus | JCM 1084 | Lactobacillus reuteri | 1.1 ± 0.00 |
| | JCM 1112 | Lactobacillus reuteri | 0.67 ± 0.02 |
| | JCM 1081 | Lactobacillus reuteri | 0.92 ± 0.06 |
| | JCM 1091 | Lactobacillus curvatus | 1.245 ± 0.02 |
| | JCM 1096 | Lactobacillus curvatus | 1.44 ± 0.1 |
| | JCM 1002 | Lactobacillus delbrueckii subsp. | N/A |
| | | Bulgaricus | |
| | JCM 1001 | Lactobacillus delbrueckii subsp. | 0.89 ± 0.02 |
| | | Bulgaricus | |
| | JCM 20398 | Lactobacillus delbrueckii subsp. | N/A |
| | | Bulgaricus | |
| | JCM 11125 | Lactobacillus plantarum/ | 1.47 ± 0.11 |
| | | arizonensis | |
| | JCM 1149 | Lactobacillus plantarum subsp. | 1.51 ± 0.21 |
| | | plantarum | |
| | JCM 6651 | Lactobacillus plantarum subsp. | 1.56 ± 0.21 |
| | | plantarum | |
| | JCM 1101 | Lactobacillus johnsonii | 1.66 ± 0.20 |
| | JCM 1096 | Lactobacillus curvatus | 1.8 ± 0.15 |
| | JCM 1091 | Lactobacillus curvatus | 1.6 ± 0.11 |
| | JCM 1133 | Lactobacillus casei subsp | 2.01 ± 0.22 |
| | | alactosus | |
| | JCM 1181 | Lactobacillus casei subsp. | 1.65 ± 0.22 |
| | | pseudoplantarum | |
| | JCM 1161 | Lactobacillus casei subsp. | 1.82 ± 0.22 |
| | | pseudoplantarum | |
| | JCM 8129 | Lactobacillus casei | 1.78 ± 0.02 |
| | JCM 1170 | Lactobacillus brevis | 1.90 ± 0.34 |
| | JCM 1059 | Lactobacillus brevis | 2.01 ± 0.27 |
| | JCM 1061 | Lactobacillus brevis | 2.06 ± 0.33 |
| | JCM 1084 | Lactobacillus reuteri | 1.22 ± 0.35 |
| | KCTC 1120 | Lactobacillus johnsonii | 1.12 ± 0.04 |
| | KCTC 3102 | Lactobacillus brevis | 1.23 ± 0.00 |
| | KCTC 3141 | Lactobacillus johnsonii | 0.41 ± 0.00 |
| | KCTC 3144 | Lactobacillus gasseri | 1.07 ± 0.04 |
| | KCTC3148 | Lactobacillus gasseri | 1.05 ± 0.02 |
| | KCTC 3163 | Lactobacillus gasseri | 1.14 ± 0.02 |
| | KCTC 3181 | Lactobacillus gasseri | 1 ± 0.02 |
| | KCTC 3237 | Lactobacillus rhamnosus | N/A |
| | | (Lactobacillus casei subsp | |
| | | rhamnosus) | |
| | KCTC 3510 | Lactobacillus paracasei subsp | 1.15 ± 0.08 |
| | | paracasei | |
| | KCTC 5049 | Lactobacillus fermentum | 0.67 ± 0.02 |
| Streptococcus | JCM20026 | Streptococcus thermophiles | 1.66 ± 0.03 |
| | JCM17834 | Streptococcus thermophilus | N/A (No clear zone) |
| Enterococcus | JCM5803 | Enterococcus faecalis | N/A (No clear zone) |
| | JCM7783 | Enterococcus faecalis | N/A (No clear zone) |
| | JCM8727 | Enterococcus faecalis | N/A (No clear zone) |
| Staphylococcus | JCM2414 | Staphylococcus epidermidis | N/A (No clear zone) |
| | JCM5692 | Staphylococcus epidermidis | N/A (No clear zone) |
| | JCM5693 | Staphylococcus epidermidis | N/A (No clear zone) |
| | JCM20345 | Staphylococcus epidermidis | N/A (No clear zone) |
| Saccharomyces | JCM1499 | Saccharomyces cerevisiae | N/A (No clear zone) |
| | JCM1817 | Saccharomyces cerevisiae | N/A (No clear zone) |
| | JCM1819 | Saccharomyces cerevisiae | N/A (No clear zone) |
| | JCM7255 | Saccharomyces cerevisiae | N/A (No clear zone) |
| Kluyveromyces | JCM5219 | Kluyveromyces lactis | N/A (No clear zone) |
| | JCM9563 | Kluyveromyces lactis | N/A (No clear zone) |
| | JCM22014 | Kluyveromyces lactis | N/A (No clear zone) |

Anti-Inflammatory Effect on Human Skin Cell

To assess the anti-inflammatory potential of probiotics as an initial step before progressing to 3D skin model studies, a skin cell investigation is conducted. In a concise summary, skin cells, including keratinocytes and fibroblasts, are cultured alongside pathogenic bacteria or fungi to elicit an inflammatory response. Various probiotics are co-inoculated to screen for promising candidates or combinations concerning their interaction with the skin cells.

The evaluation focuses on pro-inflammatory cytokines, such as TNF-α, IL-1β, IL-6, and IL-18, as well as protein and collagen levels within the keratinocytes or fibroblasts. In this specific instance, the anti-inflammatory effect of Lactobacillus probiotics is assessed using a keratinocyte cell line, HaCaT. Staphylococcus aureus serves as the pathogenic bacteria to induce inflammation in the HaCaT cells.

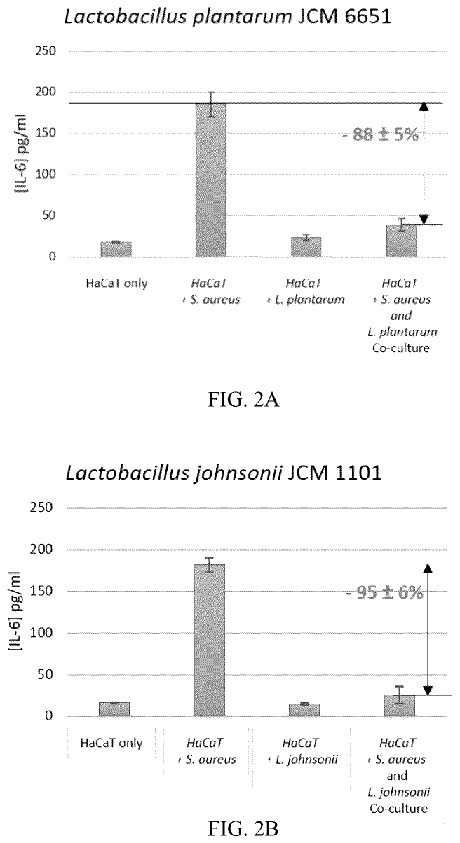

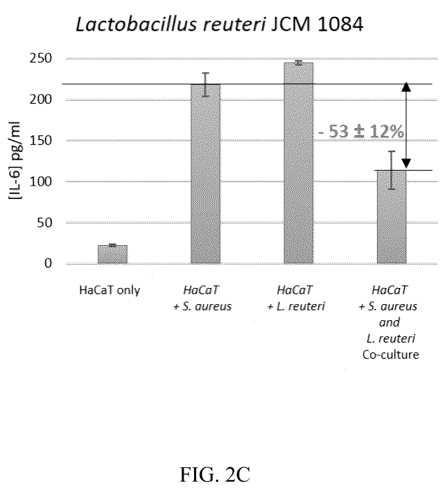

As illustrated in FIG. 2A-2B, incubating HaCaT cells with S. aureus significantly elevates IL-6 levels compared to the control group (HaCat cells only). Conversely, Lactobacillus plantarum (JCM 6651) and Lactobacillus johnsonii (JCM 1101) do not induce inflammation. Remarkably, when HaCaT cells are co-cultured with both S. aureus and Lactobacillus plantarum, IL-6 levels decrease significantly by 88-95% in comparison to cells exposed to S. aureus alone.

Conversely, as depicted in FIG. 2C, the incubation of S. aureus leads to a significant increase in IL-6 levels compared to the control group (HaCat cells only). Intriguingly, Lactobacillus reuteri alone induces notable inflammation in HaCaT cells, with the magnitude of inflammation comparable to that induced by S. aureus. However, when HaCaT cells are co-cultured with both S. aureus and Lactobacillus reuteri, the IL-6 levels decrease by 53±12% compared to cells exposed to S. aureus alone.

Anti-Inflammatory Effect on 3D Skin Model

A 3D EpiDerm skin model, also known as reconstructed human epidermis (RHE), is utilized to assess the anti-inflammatory properties of probiotics. To replicate skin conditions influenced by inflammation induced by bacteria, live pathogenic bacteria such as S. aureus are introduced onto the surface of the 3D skin model and co-cultured. Careful optimization is carried out to control the adhesion of bacteria to the model.

Following this, the selected probiotics are applied to the 3D skin model intentionally induced with bacterial inflammation. ELISA kit is employed to quantify the levels of inflammation-related cytokines, including TNF-α, IL-1β, IL-6, and IL-18, along with collagen levels. These measurements serve as crucial indicators for evaluating the probiotics' protective impact on the skin.

Subsequently, the strain of probiotics demonstrating the most pronounced antibacterial and anti-inflammatory effects is identified. This strain is chosen for the development of the core-shell particle. Moreover, its corresponding lysates are utilized in the formulation of the postbiotic formulation.

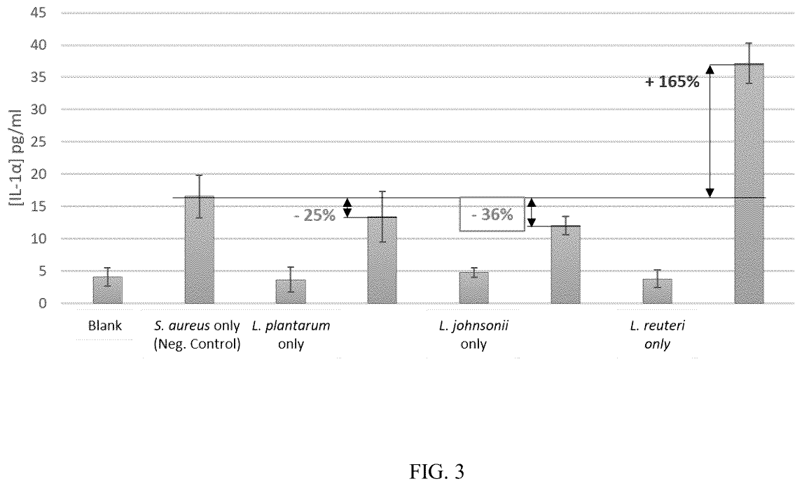

FIG. 3 presents the results of this experiment. The presence of S. aureus leads to a significant increase in IL-1α levels compared to the control group, where no S. aureus infection occurs. Conversely, Lactobacillus plantarum (JCM 6651), Lactobacillus johnsonii (JCM 1101), and Lactobacillus reuteri (JCM 1084) exhibit no signs of inflammation. When the 3D skin model is co-cultured with both S. aureus and either Lactobacillus plantarum (JCM 6651) or Lactobacillus johnsonii (JCM 1101), a noticeable reduction in IL-1α levels is observed-by 25% and 36%, respectively-compared to the scenario where only S. aureus is present. Conversely, co-culturing the 3D skin model with S. aureus and Lactobacillus reuteri (JCM 1084) prompts a significant inflammatory response, leading to a 165% increase in IL-la levels. The summarized results detailing the anti-inflammatory impact of Lactobacillus probiotics on the human 3D skin model are presented in Table 3.

TABLE 3
Summary for the anti-inflammatory effect of Lactobacillus
probiotics on human 3D skin model.

| | Batch | Batch | Batch | Average anti- |
|---|---|---|---|---|
| Lactobacillus | 1 | 2 | 3 | inflammation |
| Lactobacillus plantarum JCM6651 | 25% | 34% | 33% | 31% |
| Lactobacillus johnsonii JCM1101 | 36% | 42% | 29% | 36% |
| Lactobacillus reuteri JCM1084 | — | 4% | 28% | 11% |

The findings clearly indicate that the probiotics Lactobacillus johnsonii JCM 1101 and Lactobacillus plantarum JCM 6651 exhibit anti-inflammatory properties. They achieve this by notably diminishing the quantity of the inflammatory cytokine IL-la released from skin cells in the 3D skin model that has been infected with S. aureus. On average, Lactobacillus plantarum JCM 6651 and Lactobacillus johnsonii JCM 1101 demonstrate a reduction of 31% and 36%, respectively, in IL-1α levels.

### Example 2

The examined prebiotics encompass oligosaccharide carbohydrates, including but not limited to resistant starch, resistant dextrins, pectins, beta-glucans, fructo-oligosaccharides, inulin, lignins, and chitins. These prebiotics play a crucial role in the core-shell particle formulation, as they notably enhance the growth of the chosen probiotic strain.

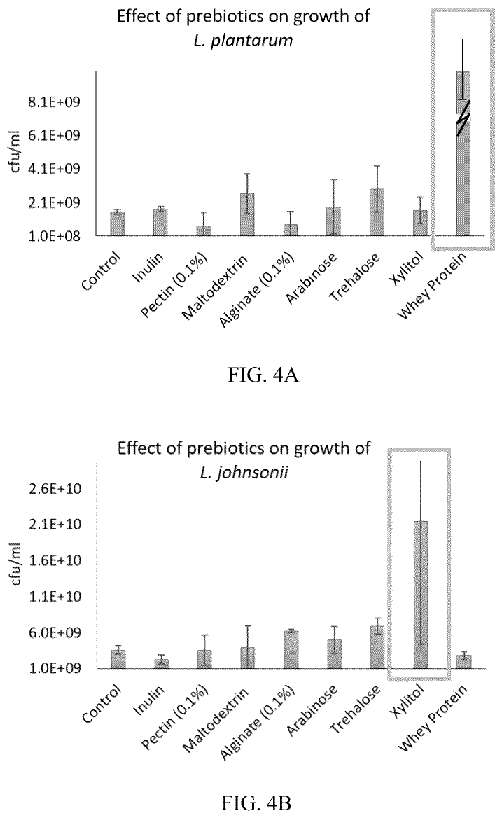

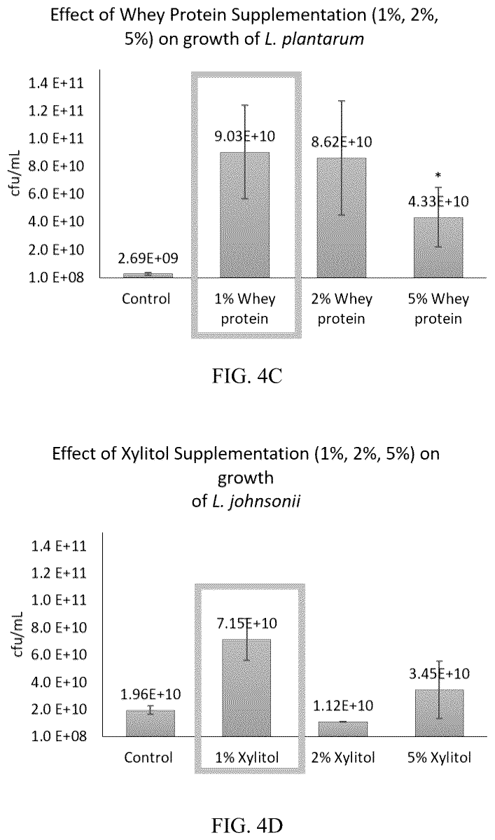

In FIG. 4A, it's evident that the addition of whey protein leads to a remarkable 21,862% increase in the growth of Lactobacillus plantarum (JCM 6651) when compared to the control group without supplementation. In contrast, the other selected prebiotics exhibit effects similar to the control group. In FIG. 4B, xylitol supplementation is shown to boost the growth of Lactobacillus johnsonii (JCM 1101) by 496% when compared to the unsupplemented control group, while the other chosen prebiotics yield effects comparable to the control. Additionally, as demonstrated in FIG. 4C, whey protein performs optimally at a concentration of 1% among all the tested doses. Similarly, xylitol demonstrates its best performance at a 1% concentration, as depicted in FIG. 4D.

### Example 3. Manufacture Procedure and Formulation of the Dormant State Encapsulated Probiotic Core-Shell Particle

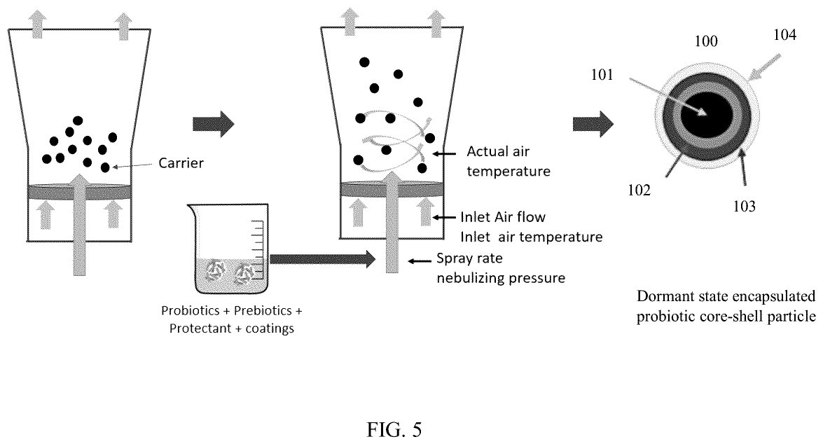

FIG. 5 provides an overview of the manufacturing process for the dormant state encapsulated probiotic core-shell particle 100. In a fluidized-bed coating process, a carrier particle core 101 serves as the initial substrate for probiotic coating. The live probiotics undergo centrifugation to eliminate the culture medium and are then blended with a solution containing monosaccharides and polysaccharides to safeguard their viability. This mixture is applied via spray-coating onto the carrier particle core 101, creating the first layer 102. Subsequently, the coating is subjected to a 30-minute drying process. Once the first layer is fully dried, a polymeric solution is spray-coated onto it, and warm air is employed to facilitate the formation of the polymer layer 103. Additionally, the dissolvable protective layer 104 is created through a spray-coating process, utilizing a distinct solution comprising polymers, surfactants, fatty acids, and minerals. The specific coating conditions, such as air flow, temperature, and coating duration, are contingent upon the desired coating thickness and the number of protective layers.

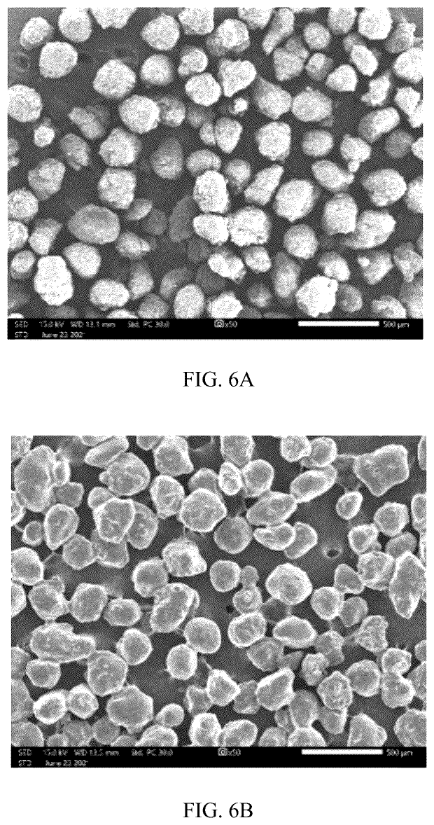

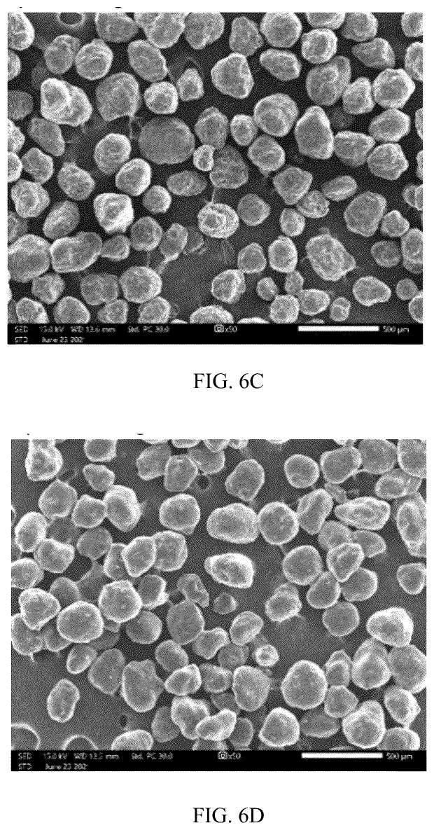

The morphology of the dormant state encapsulated probiotic core-shell particle 100 is meticulously examined using a JEM-IT200 scanning electron microscope (SEM). In this analysis, the powder's size is gauged utilizing the SEM's scale, and the powder's uniformity is assessed. To prepare the samples for examination, they are securely affixed to metal stubs using adhesive tape and rendered electrically conductive through a gold sputter-coating process. Subsequently, the samples are observed using the SEM, and the SEM images of the particles at various stages of the manufacturing process are presented in FIGS. 6A-6D. FIG. 6A illustrates the SEM image of the carrier particle core (sucrose sphere), while FIG. 6B provides a view of the carrier particle core coated with the first layer. Additionally, FIG. 6C showcases the SEM morphology of the polymer layer on the first layer's surface, and FIG. 6D reveals the SEM image of the dormant state encapsulated probiotic core-shell particle subsequent to the application of the dissolvable protective layer onto the polymer layer.

Based on the results of the above examples, Lactobacillus are chosen for the core-shell particle formulation in this example. The selected probiotics are formulated with the following chemicals to obtain a high viability, stable and easy released formulation. The formulations are listed in the Table 4.

The formulation of the core-shell particle designed to safeguard live probiotics encompasses several key components. Firstly, the seed material is carefully chosen from saccharides, which include monosaccharides, disaccharides, and polysaccharides, each varying in shape and size. The probiotics coating consists of at least one live probiotic hailing from the Lactobacillus species and at least one prebiotic, which is selected from proteins like whey protein and saccharides such as maltodextrin, xylitol, and sucrose.

Furthermore, the protective coating layers comprise at least one polymer component, chosen from options like dipalmitoyl hydroxylproline, butyl methacrylate, dimethylaminoethyl methacrylate, and methyl methacrylate copolymer. Additionally, one or more surfactants, such as sodium dodecyl sulfate, are included in the formulation. To enhance its properties, the formulation also integrates at least one fatty acid, such as stearic acid, and at least one mineral, like Talc. These components collectively contribute to the effectiveness of the core-shell particle in preserving and delivering probiotics.

TABLE 4
Composition of the dormant state encapsulated probiotic core-shell particle

| | Formulation (%) | | | | | | | | | | |
|---|---|---|---|---|---|---|---|---|---|---|---|
| | DP01 | DP03 | DP06 | DP07 | DP08 | DP09 | DP17 | DP18 | DP19 | DP20 | DP21 |
| Sucrose Sphere | 83.9 | 86.4 | 87.0 | 84.3 | 86.3 | 83.0 | 77.1 | 82.0 | 87.1 | 74.6 | 77.7 |
| Maltodextrin | 8 | 8.70 | 4.4 | 8.4 | 8.2 | 6.6 | 6.2 | 6.6 | 7.0 | 6.0 | 6.2 |
| Xylitol | — | 0.40 | 0.4 | 0.4 | 0.4 | 0.3 | 0.3 | 0.3 | 0.3 | 0.3 | 0.3 |
| Whey protein | — | 2.20 | 2.2 | 2.1 | 2.1 | 1.7 | 1.5 | 1.6 | 1.7 | 1.5 | 1.5 |
| Sucrose | — | — | 2.2 | 2.1 | 2.1 | 1.7 | 1.5 | 1.6 | 1.7 | 1.5 | 1.5 |
| Lactobacillus | 0.1 | 0.1 | 0.1 | 0.1 | 0.1 | 0.1 | 0.1 | 0.1 | 0.1 | 0.1 | 0.1 |
| Chitosan | — | 2.20 | 3.3 | — | — | — | — | — | — | — | — |
| Acetic Acid | — | — | 0.6 | — | — | — | — | — | — | — | — |
| Dipalmitoyl | — | — | — | — | — | 3.3 | 3.1 | 3.9 | — | 3.0 | 3.1 |
| Hydroxylproline | | | | | | | | | | | |
| Eudragit ® EPO | — | — | — | — | — | — | 6.4 | — | — | 9.0 | 6.2 |
| Sodium dodecyl | — | — | — | — | — | — | 0.6 | — | — | 1.3 | 0.6 |
| sulfate | | | | | | | | | | | |
| Stearic Acid | — | — | — | — | — | — | 1.0 | — | — | — | 0.9 |
| Talc | — | — | — | — | — | — | 2.2 | — | — | — | 1.9 |
| Shellac | — | — | — | 2.5 | — | 3.3 | — | 3.9 | — | — | — |
| Pectin | — | — | — | — | 0.8 | — | — | — | 0.7 | — | — |
| Calcium | — | — | — | — | — | — | — | — | 1.4 | — | — |
| Chloride | | | | | | | | | | | |
| Precirol ® | — | — | — | — | — | — | — | — | — | 2.7 | — |
| ATO 5 | | | | | | | | | | | |
| Arcyl-EZE | 8 | — | — | — | — | — | — | — | — | — | 1 |

The stability of the formulations is assessed over various time intervals, and the viability of the protected live probiotics is examined. The formulation is subjected to a releasing medium, diluted to an appropriate concentration, and subsequently inoculated onto MRS agar for 24-48 hours. The colony-forming units (CFU) are recorded and compared with the initial time point of the formulation. The results are presented in Table 5.

TABLE 5
Probiotics viability after 24 weeks

| | | Initial | | Probiotics |
|---|---|---|---|---|
| | | probiotics | Log | amount after |
| Formulation | Coating | amount (log) | Reduction | 24 weeks (log) |
| DP17 | EPO | 8.4 | 2.3 | 6.1 |
| DP18 | Shellac | 8.4 | 2.2 | 6.2 |
| DP19 | Pectin | 8.5 | 1.6 | 6.9 |
| DP20 | EPO + ATO5 | 8.5 | 3.4 | 5.1 |
| DP21 | EPO | 9.1 | 4.2 | 4.9 |

### Example 4. Stability Evaluation of the Dormant State Encapsulated Probiotic Core-Shell Particle Formulations

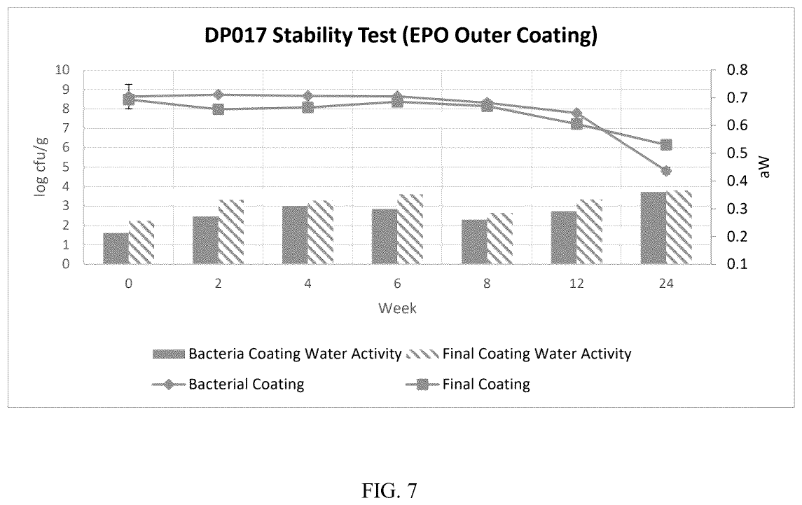

The structural conditions and a summary of the DP17 formulation are provided in Table 6. The stability of DP17 is assessed by monitoring probiotic viability at various time intervals while it is stored at room temperature. Additionally, changes in water activity are monitored over time. The stability of DP017 is illustrated in FIG. 7.

TABLE 6
Formulation conditions of DP17

| Formulation | DP17 (EPO Outer Coating) |
|---|---|
| Carrier | 77.1% Sucrose Sphere |
| Bacteria Coating | JCM1101 in PBS with 6.2% Maltodextrin + |
| | 0.3% Xylitol + 1.5% Whey |
| | protein + 1.5% Sucrose |
| 1^{st} Coating | 3.1% DPHP |
| Final Coating | 6.4% EPO + 0.6% SDS + 1.0% |
| | Stearic Acid + 2.2% Talc |
| Releasing medium | Acidic Condition (pH <5) |
| Remarks | Encapsulation efficiency: 90%; |
| | Fragment Size <10 μm |

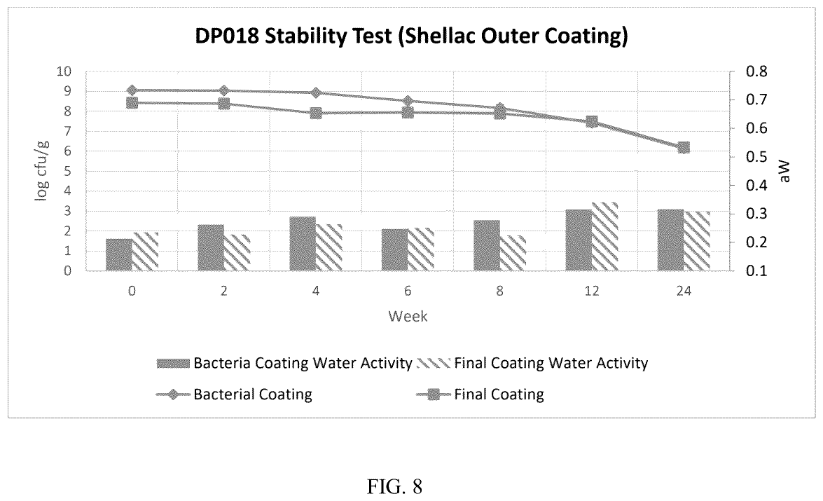

The structural conditions and a summary of the DP18 formulation are provided in Table 7. The stability of DP18 is assessed by monitoring probiotic viability at various time intervals while it is stored at room temperature. Additionally, changes in water activity are monitored over time. The stability of DP18 is illustrated in FIG. 8.

TABLE 7
Formulation conditions of DP18

| Formulation | DP18 (Shellac Outer Coating) |
|---|---|
| Carrier | 82.0% Sucrose Sphere |
| Bacteria Coating | JCM1101 in PBS with 6.6% Maltodextrin + |
| | 0.3% Xylitol + 1.6% Whey |
| | protein + 1.6% Sucrose |
| 1^{st} Coating | 3.9% DPHP |
| Final Coating | 3.9% Shellac |
| Releasing medium | Alkaline condition (pH 8) |
| Remarks | Encapsulation efficiency: 87%; |
| | Fragment Size <10 μm |

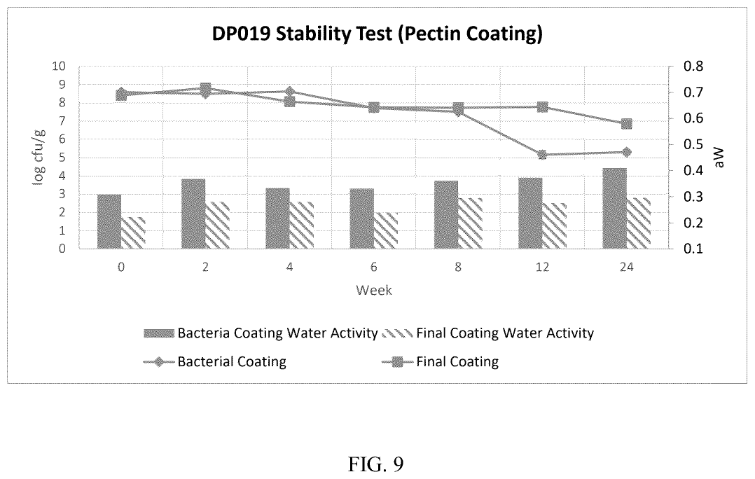

The structural conditions and a summary of the DP19 formulation are presented in Table 8. To assess the stability of DP19, the probiotic viability at various time points is monitored while it is stored at room temperature. Additionally, changes in water activity are monitored over time. The stability of DP19 is depicted in FIG. 9.

TABLE 8
Formulation conditions of DP19

| Formulation | DP19 (Pectin Outer Coating) |
|---|---|
| Carrier | 87.1% Sucrose Sphere |
| Bacteria Coating | JCM1101 in PBS with 7.0% Maltodextrin + |
| | 0.3% Xylitol + 1.7% Whey |
| | protein + 1.7% Sucrose |
| 1^{st} Coating | 0.7% Pectin |
| Final Coating | 1.4% CaCl₂ |
| Releasing medium | Acidic Condition (pH <5) |
| Remarks | Encapsulation efficiency: 88%; |
| | Fragment Size <10 μm |

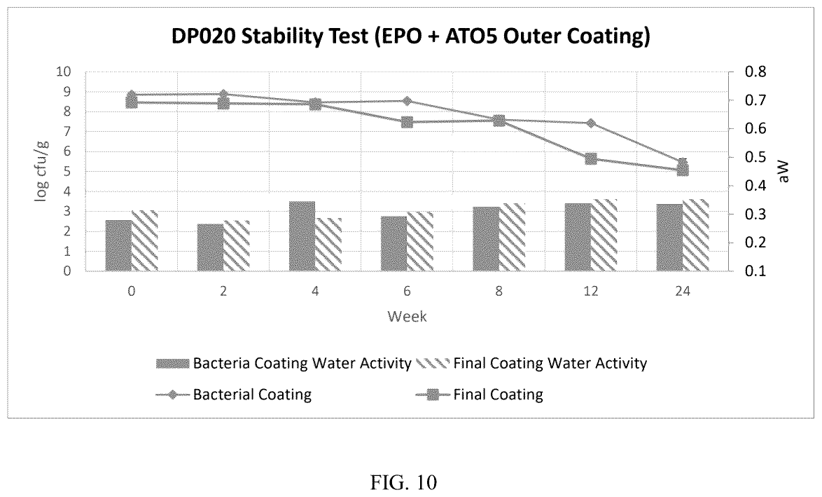

The structural conditions and a summary of the DP20 formulation are presented in Table 9. To assess the stability of DP20, the probiotic viability at various time points is monitored while it is stored at room temperature. Additionally, changes in water activity are monitored over time. The stability of DP20 is depicted in FIG. 10.

TABLE 9
Formulation conditions of DP20

| Formulation | DP20 (EPO + ATO5 Outer Coating) |
|---|---|
| Carrier | 74.6% Sucrose Sphere |
| Bacteria Coating | JCM1101 in PBS with 6.0% Maltodextrin + |
| | 0.3% Xylitol + 1.5% Whey |
| | protein + 1.5% Sucrose |
| 1^{st} Coating | 3% DPHP |
| Final Coating | 9% EPO + 1.3% SDS + 2.7% ATO5 |
| Releasing medium | Acidic Condition (pH 4.8) |
| Remarks | Encapsulation efficiency: 86%; |
| | Fragment Size <10 μm |

### Example 5. Releasing Medium Formulations

The releasing medium formulation is carefully crafted to facilitate the release and reactivation of the dormant probiotics from the core-shell particle. This formulation includes specific chemical components designed to dissolve the protective layers, which comprise both the dissolvable protective layer and the polymer layer. Additionally, it aids in the colonization of probiotics at the application site. To achieve this, organic acids are chosen to regulate the pH, promoting the dissolution of the protective layer materials. Meanwhile, film-forming nutrients like hyaluronic acid and extracellular polysaccharides support the settling of probiotic colonies on the skin. Surfactants and oils are incorporated to enhance the dissolution of the protective layers and provide improved extensibility during application. The detailed compositions of the releasing medium formulations are summarized in Table 10.

TABLE 10
Formulations of releasing medium

| Ingredients | RM09a | RM09b | RM10a | RM10b | RM10c | RM11a | RM11b | RM12 |
|---|---|---|---|---|---|---|---|---|
| Sterilized water | 98.1% | 98.1% | 98.1% | 98.1% | 98.1% | 97.6% | 97.6% | 96.7% |
| Sodium | — | — | — | — | — | — | — | 0.90% |
| Chloride | | | | | | | | |
| Lactic Acid | — | — | 0.02% | 0.02% | 0.02% | 0.02% | 0.02% | 0.01% |
| Hyaluronic | 0.20% | 0.20% | 0.20% | 0.20% | 0.20% | 0.20% | 0.20% | 0.20% |
| Acid | | | | | | | | |
| Extracellular | 0.20% | 0.20% | 0.20% | 0.20% | 0.20% | 0.20% | 0.20% | 0.20% |
| Polysaccharides | | | | | | | | |
| Tween 80 | 1% | 1% | 1% | 1% | 1% | 1% | 1% | 1% |
| Meadowfoam | — | 0.50% | — | — | — | 0.50% | 0.50% | 0.50% |
| Seed Oil | | | | | | | | |
| Coconut Oil | 0.50% | — | — | — | — | — | — | — |
| Squalane | — | — | — | 0.50% | — | — | 0.50% | 0.50% |
| Salicylic Acid | — | — | — | — | 0.50% | — | — | — |
| Allantoin | — | — | 0.50% | — | — | 0.50% | — | — |
| Release | 99% | 100% | 100% | 100% | 42% | 91% | 96% | 96% |
| probiotics | | | | | | | | |
| amount | | | | | | | | |

The preparation of the releasing medium formulation is a straightforward process, achieved through simple stir-mixing or homogenization mixing methods. To outline the procedure, 96.7 grams of water are measured into a beaker, followed by the addition of 0.9 grams of sodium chloride, which is then dissolved in the water. Subsequently, 0.01 grams of lactic acid, 0.2 grams of hyaluronic acid, and 0.2 grams of extracellular polysaccharides are carefully weighed and likewise dissolved in the water. Then, 1 gram of Tween 80 is measured and added to the mixture, which is stirred at 500 rpm for 30 minutes. Afterward, 0.5 grams each of meadowfoam seed oil and squalane are introduced into the mixture, which is further stirred at 1000 rpm for at least 30 minutes or subjected to homogenization at 5000 rpm for 5 minutes to yield the final releasing medium.

### Example 6. Protected Postbiotics Formulations

The formulation of protected postbiotics comprises carefully selected combinations of cell lysates and prebiotics, identified through prior evaluations. Probiotic cell lysates are obtained by subjecting the fermented cultures to either thermal treatment or sonication to rupture the cells. These cell lysates encompass debris from probiotic cell walls, growth metabolites, and nonviable probiotics. To safeguard their bio-functionality against microbial activity, these components are combined with saccharides. The materials employed in this formulation are outlined in Table 11.

TABLE 11
Protected postbiotics formulation summary

| | Batch No. | | | | |
|---|---|---|---|---|---|
| | LJSM02 | LJSM03 | LJSM03a | LJSM04 | LJSM05 |
| | Formulation | | | | |
| | | 20% (w/v) | 20% (w/v) | 20% (w/v) | 20% (w/v) |
| | 20% | maltodextrin + | maltodextrin + | maltodextrin + | maltodextrin + |
| | maltodextrin | 1% xylitol | Spent | 1% xylitol | 1% xylitol |
| | (w/v) + 1% | (w/v) + 5% | medium + 1% | (w/v) + 2.5% | (w/v) + 1.25% |
| | xylitol | trehalose | xylitol | trehalose | trehalose |
| | (w/v) + | (w/v) + | (w/v) + 5% | (w/v) + | (w/v) + |
| | Spent | Spent | trehalose | Spent | Spent |
| | medium | medium | (w/v) | medium | medium |
| Maltodextrin | 15-20 | 16.5-19.5 | | | |
| dextro-se | | | | | |
| equivalent | | | | | |
| (DE) | | | | | |
| *Antibacterial | 3.98 | 2.02 | 5.98 | 2.21 | 2.59 |
| effect (Log | | | | | |
| reduction at | | | | | |
| week 0) | | | | | |

The protected postbiotics formulation is meticulously prepared in powder form using the freeze-drying method. This approach preserves temperature-sensitive proteins and other bio-functional substances effectively. To outline the procedure briefly, the postbiotics are extracted from the probiotics culture solution and subsequently filtered through a membrane filter to eliminate larger debris and undesired components. The resulting solution is enriched with maltodextrin, xylitol, and trehalose. After thorough mixing for 30 minutes, the solution is promptly frozen, either in a −80° C. refrigerator or using liquid nitrogen. Subsequently, the frozen sample is transferred to a designated vacuum environment within a freeze dryer, where the ice water is removed through sublimation. The outcome of this process is the freeze-dried postbiotics, now in a convenient powder form.

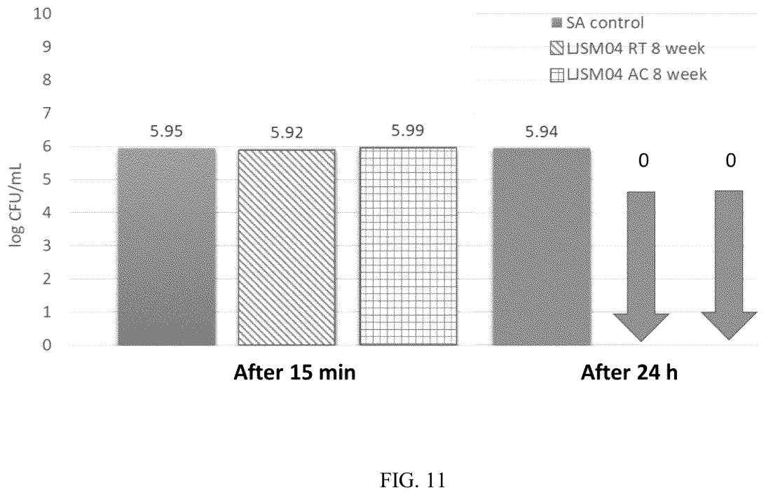

The antibacterial effectiveness of these protected postbiotics formulations is assessed by dissolving the protected postbiotics powder in a 5% concentration of phosphate-buffered saline (PBS) and testing it against Staphylococcus aureus. Specifically, 10⁶ CFU/ml of Staphylococcus aureus is introduced into the 5% protected postbiotics formulation. Sampling is conducted at two distinct time points: 15 minutes and 24 hours after contact. The results of this evaluation are presented in FIG. 11.

In Table 12, the stability of protected postbiotics is shown. The antibacterial performance against Staphylococcus aureus is reached >5 log reduction after 3 months of accelerated condition (40° C., 75% RH). The appearance of the powder is pale yellow and remains slightly change over the entire stability evaluation.

TABLE 12
Stability evaluation of protected postbiotics formulation

| | Stability Test Condition | | | |
|---|---|---|---|---|
| | Real Time | | Accelerated | |
| | (25° C., 60% RH) | | (40° C., 75% RH) | |
| | Antibacterial | | Antibacterial | |
| | Test against SA | Visual | Test against SA | Visual |
| Week | (log reduction) | Check | (log reduction) | Check |
| Week 0 | >5 | Pale yellow | / | Pale Yellow |
| Week 2 | >5 | Pale yellow | >5 | Pale Yellow |
| Week 4 | >5 | Pale yellow | >5 | Pale Yellow |
| Week 8 | >5 | Pale yellow | >5 | Pale Yellow |
| Week 12 | >5 | Pale yellow | >5 | Pale yellow |

### Example 7. The Safety Assessments of PRS and Protected Postbiotics

The PRS and protected postbiotics are submitted to accredited third-party laboratories for a comprehensive assessment of their biological safety. The results of these assessments are presented in Table 13. The PRS and postbiotics undergo testing for contact acute toxicity, repeated contact, and human patch tests. These tests provide critical information regarding potential health hazards stemming from short-term and repeated chemical exposure through the dermal route. Importantly, no or negligible contact acute toxicity is observed, indicating their safety profile.

TABLE 13
Summary of safety assessments

| | Acute Dermal | Repeated Dermal | Human Skin Closed |
|---|---|---|---|
| | Toxicity Test | Irritation Test | Patch Test |
| PRS | Pass | Pass | Pass |
| Postbiotics | Pass | Pass | Pass |

Acute dermal toxicity, Repeated Dermal Irritation Test &, Human Skin Closed Patch Test were performed in SGS.

Furthermore, the PRS and protected postbiotics also go through clinical evaluations performed by dermatologist, and the results are shown in Table 14. There is no adverse events observed.

TABLE 14
Dermatologist clinical evaluation of safety on PRS and postbiotics

| | Proportion of |
|---|---|
| Items: | adverse events |
| Adverse events observed by dermatologists | 0% (0/60) |
| Adverse events associated with the | 0% (0/60) |
| investigational sample which | |
| assessed by dermatologists | |
| serious adverse events associated | 0% (0/60) |
| with the investigational sample | |
| which assessed by dermatologists | |

### Example 8. The Probiotics Colonization and Moisturizing Effect of a PRS which is Applying the Core-Shell Particle and the Releasing Medium Together

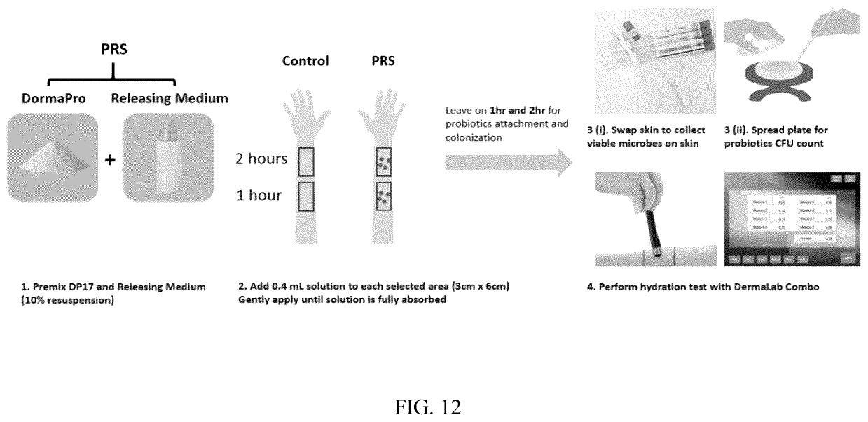

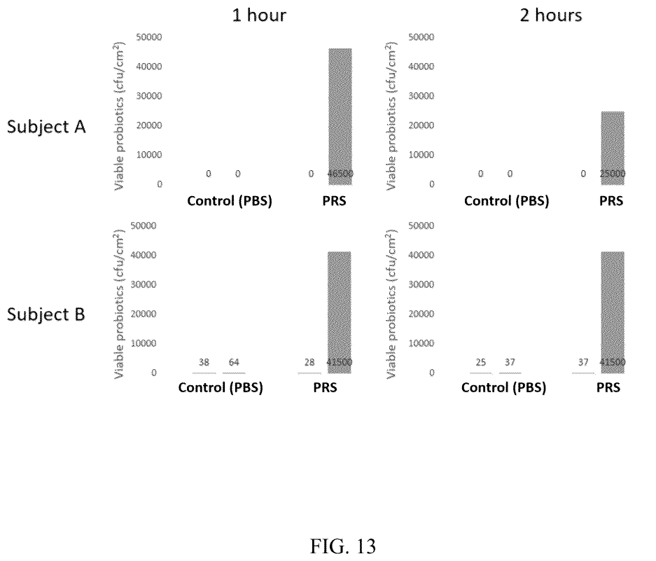

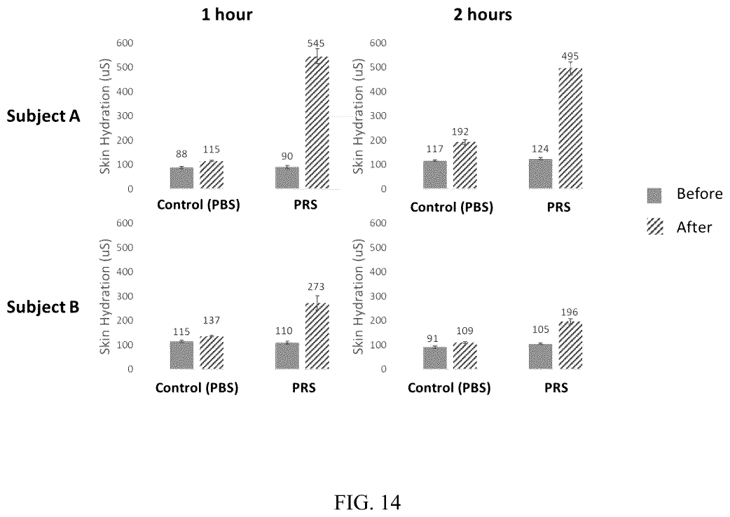

The colonization ability of the probiotics released by the PRS, achieved by mixing the core-shell particle with the releasing medium, is assessed on human skin, along with an evaluation of its skin moisturizing effect. As shown in FIG. 12, two subjects participate in the study, with one hand treated using PRS and the other serving as a control with PBS. For the PRS application, 0.1 g of the core-shell particle formulation is combined with 0.9 g of the releasing medium formulation and thoroughly mixed. A 3×6 cm area on one hand is treated with 0.4 g of this mixture, and two such areas are designated for sample collection at two time points. This process is replicated using PBS on the other hand. Probiotic samples are collected at 1 and 2-hour intervals and inoculated onto agar plates. As illustrated in FIG. 13, the amount of skin probiotics on the hand treated with PRS increases. Additionally, as shown in FIG. 14, the skin hydration level, measured at various time points, demonstrates an increase on the hand treated with PRS.

Probiotic skin colonization is also assessed through a randomized, double-blind, split-side study, conducted by an accredited third-party testing laboratory. Initially, an eleven-candidate preliminary study is undertaken to establish feasibility and determine the necessary test parameters. Subsequently, a standard study involving a minimum of 30 participants is initiated. Subjects are instructed to apply a standardized amount of PRS sample (e.g., 1 g of the core-shell particle and 1 ml of probiotics releasing medium) either on the left or right side of their forehead, face, and forearm skin, twice daily.

The probiotics' initial count on the skin surface is recorded on day 0 as the baseline. Samples are collected from the subjects on day 14, 28, and 56 to assess probiotic colonization. The results of this study are presented in Table 15 for comparison and demonstration of probiotic colonization.

TABLE 15
Summary of probiotics colonization of 30 subjects

| | 0 d 0 h | 0 d 2 h | 14 d 2 h | 28 d 2 h | 56 d 2 h |
|---|---|---|---|---|---|
| | Average | Average | Average | Average | Average |
| Test Site | CFU (Q1) | CFU (Q2) | CFU (Q3) | CFU (Q4) | CFU (Q5) |
| Forehead | 1.1 × 10⁶ | 3.9 × 10⁴ | 4.4 × 10⁴ | 3.9 × 10⁴ | 5.1 × 10⁴ |
| | | 3.5%* | 4.0%** | 3.5%# | 4.6%## |
| Cheek | 8.3 × 10⁵ | 9.5 × 10⁴ | 7.5 × 10⁴ | 6.8 × 10⁴ | 9.5 × 10⁴ |
| | | 11.0%* | 9.0%** | 8.2%# | 11.0%## |
| Arm(inside) | 3.8 × 10⁵ | 1.4 × 10⁴ | 1.3 × 10⁴ | 1.4 × 10⁴ | 1.8 × 10⁴ |
| | | 3.7%* | 3.4%** | 3.7%# | 4.7%## |

*Recovery (Q2/Q1 * 100%),
**Recovery (Q3/Q1 * 100%),
#Recovery (Q4/Q1 * 100%),
##Recovery (Q5/Q1 * 100%)

Moreover, the influence of the skin microbiome following the topical application of PRS is assessed through a randomized, double-blind, split-side study conducted by an accredited third-party testing laboratory. A total of thirty subjects are selected to participate in this study. Participants are instructed to apply a standardized amount of PRS sample, twice daily, either on the left or right side of their forehead, face, and forearm skin for a duration of 56 days.

Microorganisms present on the skin are collected at various time points: day 0 (before application, serving as the baseline), day 14, day 28, and day 56. These collected samples undergo evaluation through 16S rRNA sequencing to analyze and compare the skin microbiome ratios and variations at different time points.

TABLE 16
Summary of human skin microbiome evaluation

| Test | Class | Class | Class | Class | Class | Class | Class | Class | Class | Class | Class |
|---|---|---|---|---|---|---|---|---|---|---|---|
| Site* | 1 | 2 | 3 | 4 | 5 | 6 | 7 | 8 | 9 | 10 | 11 |
| A0 | 93.68% | 2.78% | 0.06% | 0.88% | 0.97% | 1.22% | 0.09% | 0.00% | 0.00% | 0.00% | 0.29% |
| B0 | 93.44% | 3.63% | 0.05% | 2.20% | 0.02% | 0.30% | 0.13% | 0.00% | 0.00% | 0.00% | 0.22% |
| C0 | 95.90% | 2.14% | 0.47% | 0.43% | 0.03% | 0.62% | 0.14% | 0.05% | 0.01% | 0.00% | 0.20% |
| A0 h | 27.13% | 71.61% | 0.56% | 0.32% | 0.00% | 0.09% | 0.10% | 0.03% | 0.08% | 0.00% | 0.07% |
| B0 h | 39.88% | 59.13% | 0.07% | 0.59% | 0.00% | 0.10% | 0.07% | 0.01% | 0.01% | 0.00% | 0.13% |
| C0 h | 28.64% | 70.42% | 0.13% | 0.31% | 0.00% | 0.13% | 0.09% | 0.00% | 0.01% | 0.00% | 0.25% |
| A2 h | 23.06% | 73.62% | 0.76% | 1.74% | 0.01% | 0.16% | 0.51% | 0.00% | 0.01% | 0.00% | 0.13% |
| B2 h | 29.97% | 65.40% | 1.10% | 3.10% | 0.00% | 0.12% | 0.19% | 0.00% | 0.02% | 0.00% | 0.10% |
| C2 h | 30.76% | 68.28% | 0.31% | 0.29% | 0.01% | 0.20% | 0.06% | 0.00% | 0.01% | 0.00% | 0.09% |
| A14 d | 8.20% | 88.54% | 0.10% | 1.56% | 0.03% | 1.34% | 0.08% | 0.02% | 0.01% | 0.00% | 0.12% |
| B14 d | 8.99% | 85.38% | 0.08% | 4.00% | 0.04% | 1.05% | 0.11% | 0.11% | 0.01% | 0.00% | 0.22% |
| C14 d | 16.48% | 81.05% | 0.16% | 0.34% | 0.06% | 1.54% | 0.10% | 0.01% | 0.01% | 0.00% | 0.25% |
| A28 d | 39.47% | 57.88% | 0.28% | 0.51% | 0.05% | 0.70% | 0.82% | 0.00% | 0.02% | 0.00% | 0.29% |
| B28 d | 43.65% | 50.75% | 0.59% | 2.19% | 0.26% | 0.82% | 1.45% | 0.01% | 0.04% | 0.00% | 0.25% |
| C28 d | 34.93% | 61.41% | 0.33% | 0.23% | 0.25% | 1.48% | 1.12% | 0.00% | 0.02% | 0.00% | 0.23% |
| A56 d | 3.66% | 90.60% | 0.70% | 1.55% | 0.19% | 1.11% | 1.68% | 0.01% | 0.02% | 0.00% | 0.48% |
| B56 d | 7.91% | 85.96% | 0.37% | 2.58% | 0.10% | 0.89% | 1.35% | 0.01% | 0.02% | 0.06% | 0.76% |
| C56 d | 4.79% | 91.15% | 0.38% | 0.27% | 0.18% | 0.93% | 1.64% | 0.03% | 0.05% | 0.00% | 0.57% |

Class 1: Gammaproteobacteria, Class 2: Bacilli, Class 3: Bacteroidia, Class 4: Actinobacteria, Class 5: Cyanobacteria, Class 6: Alphaproteobacteria, Class 7: Clostridia, Class 8: Fusobacteriia, Class 9: Negativicutes, Class 10: Thermoleophilia, Class 11: Others
*A: Forehead, B: Cheek, C: Arm (inside), 0: before application, 0 h: immediate after application, 2 h: 2 hours after application, 14 d: after 14 days application, 28 d: after 28 days application, 56 d: after 56 days application.

Skin microbiome changed after PRS applied on the skin. Before application, the microbiome of skin is dominated by Gammaproteobacteria (>90% amount of skin microbes). After applying PRS, skin microbiome is changed to Bacilli dominated. And this phenomenon is observed from the sample immediate collected till the last sample obtained at day 56.

### Example 9. Beneficial Effects of Applying a Kit that Includes PRS and Protected Postbiotics

PRS and the protected postbiotics undergo assessment at an accredited third-party testing laboratory to determine their efficacy on healthy, sensitive, and problematic skin. A randomized, double-blind, split-side study is conducted on a minimum of 30 subjects for each prototype (PRS and postbiotics+releasing medium) and each skin condition. The prototypes are evaluated for their impact on:
1. Healthy skin, focusing on skin elasticity and moisturizing effects.
2. Sensitive skin, with emphasis on skin irritation relief and moisturizing capabilities.
3. Problematic skin, specifically assessing acne skin improvement.

Each participant is randomly assigned to apply the test sample on either the left or right side of their forehead, face, and forearm, twice daily, for a duration of 28 days. Skin condition is evaluated at day 0, day 14, and day 28 using Visia CR and assessments by dermatologists. Additionally, participants provide feedback through a questionnaire to evaluate their experience and the performance of the prototypes. Detailed results are summarized in Tables 17 to 22.

TABLE 17
Skin elasticity effect of PRS on healthy skin

| | | Test sample | | |
|---|---|---|---|---|
| | | | Difference | |
| | | | Analysis | |
| Test | Time | Rate of | (comparing | Parameter |
| Parameter | point | change | with D 0) | Remarks |
| Skin hydration | After 14 days | 33.25% | Significant | The increase of measured |
| | application | | difference | value means the increase of |
| | After 28 days | 34.55% | Significant | skin hydration |
| | application | | difference | |
| Trans-epidermal | After 14 days | −7.47% | No significant | The decrease of measured |
| water loss | application | | difference | value means the |
| TEWL | After 28 days | −14.37% | Significant | improvement of skin barrier |
| | application | | difference | function |
| Skin elasticity | After 14 days | 15.51% | Significant | The increase of measured |
| value R2 | application | | difference | value means the increase of |
| | After 28 days | 17.51% | Significant | skin elasticity level |
| | application | | difference | |
| Skin firmness | After 14 days | −1.69% | No significant | The decrease of measured |
| value F4 | application | | difference | value means the increase of |
| | After 28 days | −11.57% | Significant | firmness level |
| | application | | difference | |

TABLE 18
Skin soothing effect of PRS on sensitive skin

| | | Test sample | | |
|---|---|---|---|---|
| | | | Difference | |
| | | | Analysis | |
| Test | Time | Rate of | (comparing | Parameter |
| Parameter | point | change | with D 0) | Remarks |
| Skin hydration | 2 hours after | 18.25% | Significant | The increase of measured |
| | application | | difference | value means the increase of |
| | 4 hours after | 25.13% | Significant | skin hydration |
| | application | | difference | |
| | After 14 days | 25.40% | Significant | |
| | application | | difference | |
| | After 28 days | 25.13% | Significant | |
| | application | | difference | |
| Trans-epidermal | 2 hours after | −11.62% | Significant | The decrease of measured |
| water loss TEWL | application | | difference | value means the |
| | 4 hours after | −18.18% | Significant | improvement of skin barrier |
| | application | | difference | function |
| | After 14 days | −11.62% | Significant | |
| | application | | difference | |
| | After 28 days | −16.16% | Significant | |
| | application | | difference | |
| Skin erythema | 2 hours after | −6.28% | Significant | The decrease of measured |
| | application | | difference | value means the decrease of |
| | 4 hours after | −5.31% | Significant | skin hemoglobin contains |
| | application | | difference | |
| | After 14 days | −10.72% | Significant | |
| | application | | difference | |
| | After 28 days | −12.39% | Significant | |
| | application | | difference | |
| Skin color | 2 hours after | −2.21% | No significant | The decrease of measured |
| (Red-green) | application | | difference | value means the less red the |
| (CM-700d) | 4 hours after | −1.47% | No significant | skin color is |
| | application | | difference | |
| | After 14 days | −10.24% | Significant | |
| | application | | difference | |
| | After 28 days | −7.86% | Significant | |
| | application | | difference | |
| Skin color | 2 hours after | −5.00% | Significant | The decrease of measured |
| (Red-green) | application | | difference | value means the less red the |
| (VISIA- | 4 hours after | −6.46% | Significant | skin color is |
| CR + IPP) | application | | difference | |
| | After 14 days | −7.49% | Significant | |
| | application | | difference | |
| | After 28 days | −9.26% | Significant | |
| | application | | difference | |
| Lactic acid | After 28 days | −26.47% | Significant | The decrease of the score |
| stinging | application | | difference | means the weaken of the |
| | | | | reaction cause by lactic acid |
| Skin redness | 2 hours after | −9.09% | Significant | The decrease of measured |
| (Dermatologist | application | | difference | value means the less red the |
| clinical | 4 hours after | −12.12% | Significant | skin color is |
| evaluation) | application | | difference | |
| | After 14 days | −12.12% | Significant | |
| | application | | difference | |
| | After 28 days | −21.21% | Significant | |
| | application | | difference | |
| Degree of skin | 2 hours after | −100.00% | No significant | The decrease of the score |
| dryness and | application | | difference | means the lower the degree |
| desquamation | 4 hours after | −100.00% | No significant | of skin dryness and |
| (Dermatologist | application | | difference | desquamation |
| clinical | After 14 days | −100.00% | No significant | |
| evaluation) | application | | difference | |
| | After 28 days | −100.00% | No significant | |
| | application | | difference | |

TABLE 19
Anti-acne effect of PRS on problematic (acne) skin

| | | Test sample | | |
|---|---|---|---|---|
| | | | Difference | |
| | | | Analysis | |
| Test | Time | Rate of | (comparing | Parameter |
| Parameter | point | change | with D 0) | Remarks |
| Skin color | After 14 days | 36.36% | Significant | The increase of measured |
| (VISIA-CR + IPP) | application | | difference | value means the skin color |
| (Instrumental | After 28 days | 57.58% | Significant | becomes lighter |
| measurement) | application | | difference | |
| Skin color | After 14 days | 18.75% | Significant | The increase of measured |
| (CM-700d) | application | | difference | value means the skin color |
| (Instrumental | After 28 days | 20.00% | Significant | becomes lighter |
| measurement) | application | | difference | |
| Papule volume | After 14 days | −9.93% | Significant | The decrease of measured |
| (Instrumental | application | | difference | value means the decrease of |
| measurement) | After 28 days | −11.35% | Significant | papule volume |
| | application | | difference | |
| Acne | After 14 days | −52.27% | Significant | The decrease of the score |
| (Dermatologist | application | | difference | means the decrease of the |
| clinical | After 28 days | −59.09% | Significant | acne number |
| evaluation) | application | | difference | |
| Papules | After 14 days | −9.46% | No significant | The decrease of the score |
| (Dermatologist | application | | difference | means the decrease of the |
| clinical | After 28 days | −63.51% | Significant | papule number |
| evaluation) | application | | difference | |
| Pustules | After 14 days | −50.00% | No significant | |
| (Dermatologist | application | | difference | |
| clinical | After 28 days | −100.00% | Significant | The decrease of the score |
| evaluation) | application | | difference | means the decrease of the |
| | | | | pustule number |
| Nodules | After 14 days | — | No significant | The decrease of the score |
| (Dermatologist | application | | difference | means the decrease of the |
| clinical | After 28 days | — | No significant | nodules number |
| evaluation) | application | | difference | |
| Total number of | After 14 days | −25.62% | Significant | The decrease of the score |
| skin lesions | application | | difference | means the decrease of the |
| (Dermatologist | After 28 days | −62.81% | Significant | total number of skin lesions |
| clinical | application | | difference | |
| evaluation) | | | | |

TABLE 20
Skin elasticity effect of protected postbiotics on healthy skin

| | | Test sample | | |
|---|---|---|---|---|
| | | | Difference | |
| | | | Analysis | |
| Test | Time | Rate of | (comparing | Parameter |
| Parameter | point | change | with D 0) | Remarks |
| Skin hydration | After 14 days | 0.90% | No significant | The increase of |
| | application | | difference | measured value means |
| | After 28 days | 10.41% | Significant | the increase of skin |
| | application | | difference | hydration |
| Trans-epidermal | After 14 days | −2.05% | No significant | The decrease of |
| water loss TEWL | application | | difference | measured value means |
| | After 28 days | −6.16% | Significant | the improvement of |
| | application | | difference | skin barrier function |
| Skin elasticity | After 14 days | −0.73% | No significant | The increase of |
| value R2 | application | | difference | measured value means |
| | After 28 days | 5.96% | Significant | the increase of skin |
| | application | | difference | elasticity level |
| Skin firmness | After 14 days | −8.67% | No significant | The decrease of |
| value F4 | application | | difference | measured value means |
| | After 28 days | −17.81% | Significant | the increase of firmness |
| | application | | difference | level |

TABLE 21
Skin soothing effect of protected postbiotics on sensitive skin

| | | Test sample | | |
|---|---|---|---|---|
| | | | Difference | |
| | | | Analysis | |
| Test | Time | Rate of | (comparing | Parameter |
| Parameter | point | change | with D 0) | Remarks |
| Skin hydration | 2 hours after | 16.67% | Significant | The increase of measured |
| | application | | difference | value means the increase |
| | 4 hours after | 14.29% | Significant | of skin hydration |
| | application | | difference | |
| | After 14 days | 11.47% | Significant | |
| | application | | difference | |
| | After 28 days | 11.69% | Significant | |
| | application | | difference | |
| Trans-epidermal | 2 hours after | −8.24% | Significant | The decrease of measured |
| water loss TEWL | application | | difference | value means the |
| | 4 hours after | −2.35% | No significant | improvement of skin |
| | application | | difference | barrier function |
| | After 14 days | −7.65% | Significant | |
| | application | | difference | |
| | After 28 days | −10.00% | Significant | |
| | application | | difference | |
| Skin erythema | 2 hours after | −3.01% | Significant | The decrease of measured |
| | application | | difference | value means the decrease |
| | 4 hours after | −2.49% | No significant | of skin hemoglobin |
| | application | | difference | contains |
| | After 14 days | −3.96% | No significant | |
| | application | | difference | |
| | After 28 days | −4.31% | Significant | |
| | application | | difference | |
| Skin color (Red- | 2 hours after | −0.16% | No significant | The decrease of measured |
| green) (CM-700d) | application | | difference | value means the less red |
| | 4 hours after | 0.54% | No significant | the skin color is |
| | application | | difference | |
| | After 14 days | −4.89% | Significant | |
| | application | | difference | |
| | After 28 days | −6.28% | Significant | |
| | application | | difference | |
| Skin color (Red- | 2 hours after | −1.56% | No significant | The decrease of measured |
| green) (VISIA- | application | | difference | value means the less red |
| CR + IPP) | 4 hours after | 1.01% | No significant | the skin color is |
| | application | | difference | |
| | After 14 days | −2.35% | No significant | |
| | application | | difference | |
| | After 28 days | −5.80% | Significant | |
| | application | | difference | |
| Lactic acid stinging | After 28 days | 41.18% | Significant | The decrease of the score |
| | application | | difference | means the weaken of the |
| | | | | reaction cause by lactic |
| | | | | acid |
| Skin redness | 2 hours after | −18.92% | Significant | The decrease of measured |
| (Dermatologist | application | | difference | value means the less red |
| clinical evaluation) | 4 hours after | −27.03% | Significant | the skin color is |
| | application | | difference | |
| | After 14 days | −18.92% | Significant | |
| | application | | difference | |
| | After 28 days | 24.32% | Significant | |
| | application | | difference | |
| Degree of skin | 2 hours after | 100.00% | No significant | The decrease of the score |
| dryness and | application | | difference | means the lower the |
| desquamation | 4 hours after | −100.00% | No significant | degree of skin dryness and |
| (Dermatologist | application | | difference | desquamation |
| clinical | After 14 days | 0.00% | No significant | |
| evaluation) | application | | difference | |
| | After 28 days | 0.00% | No significant | |
| | application | | difference | |

TABLE 22
Anti-acne effect of protected postbiotics on problematic (acne) skin

| | | Test sample | | |
|---|---|---|---|---|
| | | | Difference | |
| | | | Analysis | |
| Test | Time | Rate of | (comparing | Parameter |
| Parameter | point | change | with D 0) | Remarks |
| Skin color | After 14 days | 38.00% | Significant | The increase of measured |
| (VISIA-CR + IPP) | application | | difference | value means the skin color |
| (Instrumental | After 28 days | 44.00% | Significant | becomes lighter |
| measurement) | application | | difference | |
| Skin color | After 14 days | 5.09% | No significant | The increase of measured |
| (CM-700d) | application | | difference | value means the skin color |
| (Instrumental | After 28 days | 12.96% | Significant | becomes lighter |
| measurement) | application | | difference | |
| Papule volume | After 14 days | −5.33% | Significant | The decrease of measured |
| (Instrumental | application | | difference | value means the decrease |
| measurement) | After 28 days | −10.67% | Significant | of papule volume |
| | application | | difference | |
| Acne | After 14 days | −29.59% | No significant | The decrease of the score |
| (Dermatologist | application | | difference | means the decrease of the |
| clinical | After 28 days | −77.55% | Significant | acne number |
| evaluation) | application | | difference | |
| Papules | After 14 days | −17.14% | Significant | The decrease of the score |
| (Dermatologist | application | | difference | means the decrease of the |
| clinical | After 28 days | −25.71% | Significant | papule number |
| evaluation) | application | | difference | |
| Pustules | After 14 days | 100.00% | No significant | |
| (Dermatologist | application | | difference | |
| clinical | After 28 days | −100.00% | No significant | The decrease of the score |
| evaluation) | application | | difference | means the decrease of the |
| | | | | pustule number |
| Nodules | After 14 days | — | No significant | The decrease of the score |
| (Dermatologist | application | | difference | means the decrease of the |
| clinical | After 28 days | — | No significant | nodules number |
| evaluation) | application | | difference | |
| Total number of | After 14 days | −25.93% | Significant | The decrease of the score |
| skin lesions | application | | difference | means the decrease of the |
| (Dermatologist | After 28 days | −64.44% | Significant | total number of skin |
| clinical | application | | difference | lesions |
| evaluation) | | | | |

Overall, the satisfaction rate of both PRS and protected postbiotics are 87.9% & 96.9% on healthy skin, 100% & 100% on sensitive skin, and 86.7% & 90.3% on problematic (acne) skin, respectively. All performance studies showed positive reinforcement of PRS and postbiotics on different type of skin evaluated by both instrument and dermatologist in the human trial tests.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. An inedible and dry dormant state encapsulated probiotic core-shell particle for external, non-mucosal skin application, wherein the particle comprises:
a carrier particle core for serving as a nutrient source for probiotics;
a first layer including a dormant probiotic, the first layer surrounding the carrier particle core and comprising at least one dormant probiotic species for affecting a non-mucosal, external epidermal biome and at least one prebiotic as a food source for the probiotic, the dormant probiotic being reconstitutable to a live probiotic when activated by a releasing medium on a non-mucosal epidermal surface;
a polymer layer positioned over the first layer, and
a dissolvable protective layer for protecting the probiotic core-shell particle from oxidation, heat and humidity, the dissolvable protective layer being dissolvable by the releasing medium to release the first layer for reconstitution.

2. The particle of claim 1, wherein the carrier particle core includes one or more of sucrose, whey protein, starch and cellulose.

3. The particle of claim 1, wherein the at least one probiotic species comprises Bifidobacterium, Lactobacillus, Lactococcus, Leuconostoc, Streptococcus, Enterococcus, Staphylococcus, Saccharomyces, Kluyveromyces, and the strain variants thereof.

4. The particle of claim 1, wherein the at least one prebiotic is selected from a protein or a saccharide, wherein the saccharide comprises a polysaccharide, a monosaccharide or a disaccharide.

5. The particle of claim 4, wherein the protein is selected from a whey protein, a casein protein, a soy protein, a milk protein, a pea protein, a rice protein, a zein, or a bovine serum albumin.

6. The particle of claim 4, wherein the saccharide is selected from dextrose, fructose, galactose, sucrose, lactose, maltose, trehalose, cellobiose, chitobiose, dextrin, maltodextrin, cyclodextrin, xylitol, cellulose, chitin, chitosan, pullulan, pectin, alginates or arabinoxylans.

7. The particle of claim 1, wherein the polymer layer is selected from shellac, dipalmitoyl hydroxylproline, a methacrylate-based polymer or copolymer, a glyceride, or poly-L-lactic acid.

8. The particle of claim 1, wherein the protective layer comprises a polymer, a surfactant, a fatty acid and a mineral.

9. The particle of claim 8, wherein the surfactant is selected from an anionic surfactant or a non-ionic surfactant.

10. The particle of claim 8, wherein the fatty acid is selected from palmitic acid, stearic acid, oleic acid, linoleic acid, arachidonic acid, eicosapentaenoic acid or docosahexaenoic acid.

11. The particle of claim 8, wherein the mineral is selected from talc, kaolin, ZnO, TiO₂ or SiO₂.

12. A topically applied kit for modulating a microbiome of a non-mucosal epidermis area, comprising:
a plurality of the dormant, encapsulated probiotic core-shell particles of claim 1; and
a releasing medium, for reconstituting the dormant, encapsulated probiotic core-shell particles,
wherein the releasing medium is configured to degrade the protective layer and the polymer layer of the dormant encapsulated probiotic core-shell particles to convert the dormant probiotic to an activated, live probiotic; and
wherein the releasing medium forms a synthetic biofilm including the activated, live probiotic on the non-mucosal epidermis providing a microenvironment for probiotics colonization.

13. The kit of claim 12, wherein the releasing medium comprises water, a salt, an organic acid, a surfactant, an oil and a film forming nutrient.

14. The kit of claim 13, wherein the salt is selected from NaCl or CaCl₂.

15. The kit of claim 13, wherein the organic acid is selected from an acetic acid, a lactic acid or a citric acid.

16. The kit of claim 13, wherein the surfactant is selected from is selected from an anionic surfactant or a non-ionic surfactant.

17. The kit of claim 13, wherein the oil is selected from a squalane, a meadowfoam seed oil, a soybean oil, an isopropyl myristate, an isopropyl palmitate, a paraffin oil, an almond oil or a soybean oil.

18. The kit of claim 13, wherein the film forming nutrient is selected from a hyaluronic acid or an extracellular polysaccharide.

19. The kit of claim 12, wherein the releasing medium is selected from a lotion form, a cream form, a serum form, or a solution form.

20. The kit of claim 12, wherein the releasing medium further includes a postbiotics agent possessing antibacterial effect and anti-inflammatory property.

21. The kit of claim 20, wherein the postbiotics agent comprises a lysate or a ferment of the probiotic same as the vehicle, wherein the postbiotics agent contains probiotic cell wall debris, growth metabolites, and dead probiotic cell.

22. A method of maintaining skin health by modulating skin microbiome, comprising: topically applying the kit of claim 12 to a non-mucosal external epidermal area in need thereof.

23. The method of claim 22, wherein the skin area in need thereof suffers from inflammation, dehydration, acne, infection, and reddening.

24. A method for manufacturing inedible and dry dormant state encapsulated probiotic core-shell particles for external, non-mucosal skin application, comprising:
preparing a carrier particle core comprising one or more nutrient sources for probiotics;
coating the carrier particle core with a first layer comprising dormant probiotics and at least one prebiotic, forming a core-shell structure;
applying a polymer layer over the first layer;
depositing a dissolvable protective layer over the polymer layer; and
drying and conditioning the resulting particles to create inedible and dry dormant state encapsulated probiotic core-shell particles.
